(19) United States
(12) Patent Application Publication
(10) Pub. No.: US 2025/0152533 A1
(43) Pub. Date: May 15, 2025

(54) Novel Beta-Alanine Compositions and Methods of Use Thereof

(71) Applicant: Natural Alternatives International, Inc., Carlsbad, CA (US)

(72) Inventors: Sreeraj Gopi, Chalakudy (IN); Di Tan, Valley Center, CA (US)

(21) Appl. No.: 18/916,596

(22) Filed: Oct. 15, 2024

Related U.S. Application Data
(60) Provisional application No. 63/548,507, filed on Nov. 14, 2023.

(51) Int. Cl. A61K 31/197; A61K 9/107; A61K 31/015; A61K 31/522; A61K 45/06; A61K 47/24; A61P 21/00; A61P 25/28

(52) U.S. Cl. CPC A61K 31/197; A61K 9/107; A61K 31/015; A61K 31/522; A61K 45/06; A61K 47/24; A61P 21/00; A61P 25/28

(57) ABSTRACT

Disclosed herein are novel beta-alanine compositions for increasing athletic performance, improving cognitive function, and attenuating the aging process. Compositions described herein can be administered to subjects, such as elderly subject, in functional or medical foods, along with other formulations to aid in healthy aging including reduced muscle wasting, improved mobility, improved cognitive performance, and the like. The compositions described herein comprise improved bioavailability and reduced paresthesia effects compared to free beta-alanine. Also described herein are methods of making the novel beta-alanine compositions disclosed herein.

### CROSS-REFERENCE TO RELATED APPLICATIONS

This Application claims the benefit of U.S. Provisional Application No. 63/548,507 filed Nov. 14, 2023. The contents of which are herein incorporated by reference in its entirety.

### BACKGROUND

Carnosine can maintain the acid-base equilibrium in several forms of tissue and is synthesized from beta-alanine via the ATP-Grasp Domain-containing protein 1, which is expressed in skeletal muscle, brain, and cardiac muscle. While ingested carnosine cannot enter cells to a significant extent, beta-alanine can enter into cells and be taken up by tissues expressing ATP-Grasp Domain-containing protein 1 enzyme. As such, supplementation of beta-alanine can decrease muscle fatigue and enhance exercise performance by increasing carnosine concentrations in skeletal muscle, which in turn reduces/clears lactate. In addition, supplementation of beta-alanine can increase carnosine concentrations in heart muscle, brain, and cells. Increased carnosine concentrations in brain can reduce brain inflammation and have exhibited various cognitive benefits, including improved expression of brain-derived neurotrophic factor in the hippocampus under stress, reduced inflammatory response to stress via reduced glial fibrillary acidic protein levels, and maintenance of dendritic complexity, which leads to healthy brain function and healthy mood balance (such as better cognitive function and reduced anxiety). Moreover, carnosine can help macrophages eliminate senescent skin cells by increasing their phagocytic capacity, can positively regulate macrophage and microglial functions, has anti-inflammatory property, and is an antioxidant, a chelator of toxic metal ions, an anti-glycating agent, and an aldehyde/carbonyl binder and thus, increased carnosine concentrations in muscle (including heart muscle) and cells can promote and maintain heart health, muscle health, bone health, and a healthy immune system, have anti-aging effects, and support general well-being including maintaining a healthy level of muscle mass and a healthy level of physical performance. Furthermore, starting as early as age 30, carnosine levels decline and the rate of decline accelerates over time during aging. Restoring carnosine levels can slow down the aging process, promote healthy aging, and support general wellness. Accordingly, improving the bioavailability of beta-alanine is believed to lead to improvements in these effects.

Declined cognitive function is common among the elderly. Increasing age is often accompanied by a progressive decline in most physiological functions such as brain, heart, bone, joint, and muscle functions and a weakened immune system. Reducing the effects of lactate buildup and fatigue can also improve physical endurance, physical performance, athletic performance, and mental acuity.

Physical exercise is vital to human health. Regular physical activity can improve muscle strength, boost endurance, and reduce the risk of developing dementia, among other diseases. However, exercise may lead to muscle fatigue due to lactate building up in skeletal muscle and muscle inflammation. Intensive exercise can also lead to declined cognitive function, which is associated with increased oxidative stress and neuroinflammation in brain. Beta-alanine supplementation can increase muscle carnosine concentrations, but prior art compositions have either led to undesirable feelings of paresthesia or lacked adequate carnosine-concentration increases.

A historical problem with the administration of beta-alanine is the sensation often experienced by users referred to as paresthesia. Paresthesia was known to present as a tingling sensation that was experienced by some users at higher doses, which were administered for beta-alanine's carnosine-boosting effects.

Therefore, to fully utilize the benefits of beta-alanine and the corresponding cellular production of carnosine, there is a need to develop improved beta-alanine compositions with increased bioavailability that enhance exercise performance, promote and maintain brain health and mood (e.g., improve cognitive function and reduce anxiety), maintain and promote heart health, muscle health, bone health, and a healthy immune system, support general well-being, and attenuate the aging process.

### SUMMARY

Embodiments of the present disclosure are based on novel beta-alanine compositions having unexpectedly increased bioavailability leading to increased cellular carnosine concentration and as a result, improved cognitive function, reduced anxiety, and improved anti-aging effects (including maintaining a healthy level of muscle mass, a healthy level of physical performance, healthy heart, healthy blood sugar levels, healthy immune system, and healthy bones and joints) improved athletic performance (including a reduction in the onset of muscle fatigue and improved endurance and working capacity). In certain embodiments, novel beta-alanine-containing compositions unexpectedly exhibited significantly reduced paresthesia effect or no paresthesia effect.

Described herein are novel beta-alanine compositions, methods of making the compositions, and methods of using the compositions.

These and other features, aspects, and advantages of the present embodiments will become understood with reference to the following description and appended claims.

### BRIEF DESCRIPTION OF THE DRAWINGS

FIG. 1A shows particle sizes and a particle distribution of an exemplary composition as described here.

FIG. 1B shows the zeta potential of particles of an exemplary composition as described herein.

FIGS. 1C (i)-(iii) show SEC images of particles of an exemplary composition as described herein and FIG. 1C (iv) shows a particle size distribution of an exemplary composition as described herein.

FIG. 2 shows cytotoxicity of Composition 2, as described herein, compared to a Reference Product, as described herein.

FIG. 3 shows cell permeability of beta-alanine from Composition 2, as described herein, compared to a Reference Product, as described herein.

FIG. 4 shows the mean plasma beta-alanine concentrations of a composition described herein that contains 400 mg of beta-alanine in subjects compared to those of two reference products, one containing 400 mg of conventional beta-alanine and the other containing 1200 mg of conventional beta-alanine in the same subjects in a double-blinded, randomized, single dose, three-treatment, and three-way crossover study.

FIG. 5 shows the mean VAS pain score recorded by subjects administered with a composition described herein that contains 400 mg of beta-alanine compared to those recorded by the same subjects administered with a reference product containing 400 mg of conventional beta-alanine and the same subjects administered with another reference product containing 1200 mg of conventional beta-alanine in a double-blinded, randomized, single dose, three-treatment, and three-way crossover study.

FIG. 6 shows the mean QLSI score recorded by subjects administered with a composition described herein that contains 400 mg of beta-alanine compared to those recorded by the same subjects administered with a reference product containing 400 mg of conventional beta-alanine and the same subjects administered with another reference product containing 1200 mg of conventional beta-alanine in a double-blinded, randomized, single dose, three-treatment, and three-way crossover study.

FIGS. 7A-7D show the mean POMS score recorded by subjects administered with a composition described herein that contains 400 mg of beta-alanine compared to those recorded by the same subjects administered with a reference product containing 400 mg of conventional beta-alanine and the same subjects administered with another reference product containing 1200 mg of conventional beta-alanine in a double-blinded, randomized, single dose, three-treatment, and three-way crossover study.

FIG. 8 shows the mean QDSA score recorded by subjects administered with a composition described herein that contains 400 mg of beta-alanine compared to those recorded by the same subjects administered with a reference product containing 400 mg of conventional beta-alanine and the same subjects administered with another reference product containing 1200 mg of conventional beta-alanine in a double-blinded, randomized, single dose, three-treatment, and three-way crossover study.

FIG. 9 shows the mean QDSA pain intensity score recorded by subjects administered with a composition described herein that contains 400 mg of beta-alanine compared to those recorded by the same subjects administered with a reference product containing 400 mg of conventional beta-alanine and the same subjects administered with another reference product containing 1200 mg of conventional beta-alanine in a double-blinded, randomized, single dose, three-treatment, and three-way crossover study.

FIGS. 10A and 10B show the mean STAI score recorded by subjects administered with a composition described herein that contains 400 mg of beta-alanine compared to those recorded by the same subjects administered with a reference product containing 400 mg of conventional beta-alanine and the same subjects administered with another reference product containing 1200 mg of conventional beta-alanine in a double-blinded, randomized, single dose, three-treatment, and three-way crossover study.

FIG. 11 shows the mean VAS scores recorded by subjects administered with a composition described herein that contains 400 mg of beta-alanine at Visit 1 and Visit 2 and those recorded by subjects administered with a reference product containing 400 mg of conventional beta-alanine at Visit 1 and Visit 2.

FIG. 12 shows the mean QLSI scores recorded by subjects administered with a composition described herein that contains 400 mg of beta-alanine at Visit 1 and Visit 2 and those recorded by subjects administered with a reference product containing 400 mg of conventional beta-alanine at Visit 1 and Visit 2.

FIGS. 13A-13D show the mean POMS scores recorded by subjects administered with a composition described herein that contains 400 mg of beta-alanine at Visit 1 and Visit 2 and those recorded by subjects administered with a reference product containing 400 mg of conventional beta-alanine at Visit 1 and Visit 2.

FIG. 14 shows the mean QDSA scores recorded by subjects administered with a composition described herein that contains 400 mg of beta-alanine at Visit 1 and Visit 2 and those recorded by subjects administered with a reference product containing 400 mg of conventional beta-alanine at Visit 1 and Visit 2.

FIG. 15 shows the mean QDSA pain intensity scores recorded by subjects administered with a composition described herein that contains 400 mg of beta-alanine at Visit 1 and Visit 2 and those recorded by subjects administered with a reference product containing 400 mg of conventional beta-alanine at Visit 1 and Visit 2.

FIGS. 16A and 16B show the mean STAI scores recorded by subjects administered with a composition described herein that contains 400 mg of beta-alanine at Visit 1 and Visit 2 and those recorded by subjects administered with a reference product containing 400 mg of conventional beta-alanine at Visit 1 and Visit 2.

### DETAILED DESCRIPTION

Described herein are novel compositions comprising one or more of beta-alanine and one or more agents selected from the group consisting of an anti-inflammatory agent and an amphiphilic agent. As used herein, beta-alanine can refer to free beta-alanine, an ester of free beta-alanine, a salt of free beta-alanine, or a combination thereof. Free beta-alanine can refer to beta-alanine having the chemical name of 3-amino propanoic acid, which is free of chemical modifications, is unencapsulated, and is not beta-alanine derivatives. Examples of beta-alanine esters include, but are not limited to beta-alanine methyl ester, beta-alanine ether ester, beta-alanine propyl ester, beta-alanine isopropyl ester, beta-alanine butyl ester, beta-alanine tert-butyl ester, and the like. Examples of the beta-alanine salts include, but are not limited to beta-alanine sulfate, beta-alanine citrate, beta-alanine maleate, beta-alanine acetate, beta-alanine oxalate, beta-alanine hydrochloride, beta-alanine hydrobromide, beta-alanine hydroiodide, beta-alanine nitrate, beta-alanine bisulfate, beta-alanine phosphate, beta-alanine isonicotinate, beta-alanine lactate, beta-alanine salicylate, beta-alanine tartrate, beta-alanine oleate, beta-alanine tannate, beta-alanine pantothenate, beta-alanine bitartrate, beta-alanine ascorbate, beta-alanine succinate, beta-alanine gentisate, beta-alanine fumarate, beta-alanine gluconate, beta-alanine glucoronate, beta-alanine glycinate, beta-alanine saccharate, beta-alanine formate, beta-alanine benzoate, beta-alanine glutamate, beta-alanine methanesulfonate, beta-alanine ethanesulfonate, beta-alanine benzenesulfonate, beta-alanine p-toluenesulfonate, and beta-alanine pamoate (i.e., 1,1′-methylene-bis-(2-hydroxy-3-naphthoate)), and the like.

In some embodiments, beta-alanine can be micronized prior to incorporation into the composition so that the composition has an improved particle size distribution over prior art beta-alanine compositions.

An anti-inflammatory agent, as described herein, may refer to an agent capable of reducing inflammation. Without being bound by any particular theory, it is believed that an anti-inflammatory agent can aid in reducing paresthesia associated with beta-alanine consumption when used in combination with the improved beta-alanine compositions described herein. Examples of the anti-inflammatory agent include, but are not limited to hydrocarbon compounds, terpenes, terpenoids, vitamins, hormones, curcumin, fish oil, ginger, resveratrol, spirulina, bromelain, green tea extract, garlic, and amino acids. In certain embodiments, the anti-inflammatory agent is beta-caryophyllene, i.e., a bicyclic sesquiterpene having the structure:

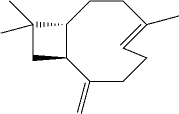

and the chemical name of (1R,4E,9S)-4,11,11-trimethyl-8-methylidenebicycol[7.2.0]undec-4-ene.

Beta-caryophyllene can be obtained from plant-derived oleoresins, essential oils, solutes, distillates, extracts, fermentations, infusions, and leachate from several plants. In certain embodiments, beta-caryophyllene is obtained from essential oils. Examples of the essential oils include, but are not limited to black pepper (Piper nigrum) oil, clove (Syzygium aromaticum) oil, cannabis (Cannabis sativa flower) oil, rosemary oil, hops oil, Cinnamomum tamala oil, Cinnamomum zeylanicum oil, Ocimum micranthum oil, Carum nigrum oil, Lavandula angustifolia oil, Cananga Odorata oil, Humulus lupulus oil, Ocimum gratissimum oil, Origanum vulgare oil, Rosmarinus officinalis oil, Sinularia nanolobata (soft coral) oil, Hyptis fruticose (leaf) oil, Eremanthus erythropappus oil, Senecio rufinervis oil, Baccharis uncinella oil, Copaifera spp. oil, Pterodon emarginatus oil, Aegle marmelos oil, Vitex agnus-castus oil, Ocimum basillicum oil, Valeriana officinalis oil, Thymus vulgaris oil, Salvia Officinalis oil, Zingiber Officinale oil, and the like. In certain embodiments, beta-caryophyllene can be obtained from the stems and/or flowers of clove by steam distillation. The stems and flowers can be dry or fresh. In certain embodiments, beta-caryophyllene is obtained by the following process: dispensing fresh clove (stems and/or flowers) in water, loading the resultant dispensation in a steam distillation unit, steam-distilling the dispensation to give a mixture of oil and water, collecting the mixture, separating the oil from water to give clove oil, transferring the clove oil to a fractional distillation column, and distilling the clove oil to give beta-caryophyllene having at least 98% purity. In certain embodiments, essential oils containing beta-caryophyllene can be used directly without the processing set forth herein. In certain embodiments, clove oil containing between about 15 wt. % and about 25 wt. % of beta-caryophyllene is used. In certain embodiments, clove oil containing about 20 wt. % of beta-caryophyllene is used.

An amphiphilic agent, as described herein, may refer to a compound having a hydrophilic group at one end and a lipophilic group at the other end and thus possessing both hydrophilic and lipophilic properties. In certain embodiments, an amphiphilic agent can form a liposome that encapsulates beta-alanine, as described herein. Without being bound by a particular theory, it is believed that the hydrophilic group of the amphiphilic agent can interact with beta-alanine by dipole-dipole and/or ion-dipole forces to form an inner core and the lipophilic group of the amphiphilic agent to form an outer shell to surround the inner core. Without being bound by a particular theory, it is believed that the lipophilic shell can pass through small intestine walls due to its lipophilic nature, which when encapsulating beta-alanine, can aid in the passage of beta-alanine through the small intestine walls where beta-alanine can then be absorbed. Without being bound by a particular theory, it is believed that the lipophilic shell can then pass through cell membranes due to its lipophilic nature as well and carry beta-alanine into the cells, which is a route for beta-alanine to pass through cell membranes in addition to a route via two transporters, i.e., TauT and PAT1. With this additional route, permeability of beta-alanine may be increased and as a result, the amount of beta-alanine in cells increases, which in turn leads to increased bioavailability of beta-alanine and increases in carnosine concentration in the cells. Without being bound by a particular theory, it is believed that such different routes of absorption due to beta-alanine being encapsulated in a liposome can be helpful in reducing paresthesia associated with beta-alanine consumption. Examples of the amphiphilic agents include, but are not limited to phospholipids, cholesterol, glycolipids, fatty acids, bile acids, saponins, and the like. In certain embodiments, an amphiphilic agent, as described herein, is a phospholipid. Phospholipids from both natural and synthetic sources are compatible with compositions described herein and may be used in varying concentrations. Examples of the phospholipids include, but are not limited to dipalmitoylphosphatidylcholine, distearoylphosphatidylcholine, diarachidoylphosphatidylcholine, dibehenoylphosphatidylcholine, diphosphatidyl glycerol, phosphatidylcholines, phosphatidylethanolamines, phosphatidylserines, phosphatidylglycerols, sphingomyelin, lecithin, phosphatidylinositols, and the like. In certain embodiments, the phospholipid, as described herein, is lecithin.

In certain embodiments, a composition, as described herein, may comprise one or more of beta-alanine and an anti-inflammatory agent. In certain embodiments, a composition, as described herein, can comprise an effective amount of beta-alanine and an effective amount of an anti-inflammatory agent. In certain embodiments, a composition, as described herein, can comprise at least about 1 wt. % of beta-alanine, as described herein, and at most about 99 wt. % of an anti-inflammatory agent, as described herein. Certain compositions can comprise between about 10 wt. % and about 99 wt. % of beta-alanine, as described herein, and between about 0.01 wt. % and about 50 wt. % of an anti-inflammatory agent, as described herein. In certain embodiments, a composition, as described herein, can comprise between about 50 wt. % and about 80 wt. % of beta-alanine, as described herein, and between about 0.01 wt. % and about 10 wt. % of an anti-inflammatory agent, as described herein. In certain embodiments, a composition, as described herein, may comprise between about 70 wt. % and about 80 wt. % of beta-alanine, as described herein, and between about 0.1 wt. % and about 4 wt. % of an anti-inflammatory agent, as described herein. In certain embodiments, a composition, as described herein, may comprise between about 70 wt. % and about 73 wt. % of beta-alanine, as described herein, and between about 0.5 wt. % and about 1 wt. % of an anti-inflammatory agent, as described herein. In certain embodiments, the amount of beta-alanine and the amount of an anti-inflammatory agent in a composition, as described herein, may be present in a weight ratio ranging from 150:1 to 1:150. In certain embodiments, the amount of beta-alanine and the amount of an anti-inflammatory agent in a composition, as described herein, can be present in a weight ratio ranging from 110:1 to 25:1.

In certain embodiments, a composition, as described herein, may comprise one or more of beta-alanine and an amphiphilic agent. In certain embodiments, a composition, as described herein, can comprise an effective amount of beta-alanine and an effective amount of an amphiphilic agent. In certain embodiments, a composition, as described herein, can comprise at least about 1 wt. % of beta-alanine, as described herein, and at most about 99 wt. % of an amphiphilic agent, as described herein. In certain embodiments, a composition, as described herein, can comprise between about 10 wt. % and about 99 wt. % of beta-alanine, as described herein, and between about 0.01 wt. % and about 50 wt. % of an amphiphilic agent, as described herein. In certain embodiments, a composition, as described herein, may comprise between about 50 wt. % and about 80 wt. % of beta-alanine, as described herein, and between about 0.1 wt. % and about 10 wt. % of an amphiphilic agent, as described herein. In certain embodiments, a composition, as described herein, can comprise between about 70 wt. % and about 80 wt. % of beta-alanine, as described herein, and between about 1 wt. % and about 4 wt. % of an amphiphilic agent, as described herein. In certain embodiments, the amount of beta-alanine and the amount of an amphiphilic agent in a composition, as described herein, may be present in a weight ratio ranging from 50:1 to 1:50. In certain embodiments, the amount of beta-alanine and the amount of an anti-inflammatory agent in a composition, as described herein, can be present in a weight ratio ranging from 50:1 to 1:1. In certain embodiments, the amount of beta-alanine and the amount of an amphiphilic agent in a composition, as described herein, may be present in a weight ratio ranging from 25:1 to 1:1. In certain embodiments, the amount of beta-alanine and the amount of an amphiphilic agent in a composition, as described herein, can be present in a weight ratio ranging from 25:1 to 10:1.

In certain embodiments, a composition, as described herein, can comprise one or more of beta-alanine, an anti-inflammatory agent, and an amphiphilic agent. In certain embodiments, a composition, as described herein, can comprise an effective amount of beta-alanine, an effective amount of an anti-inflammatory agent, and an effective amount of an amphiphilic agent. In certain embodiments, a composition, as described herein, can comprise at least about 1 wt. % of beta-alanine, as described herein, at most about 99 wt. % of an anti-inflammatory agent, as described herein, and at most about 99 wt. % of an amphiphilic agent, as described herein. In certain embodiments, a composition, as described herein, can comprise between about 10 wt. % and about 99 wt. % of beta-alanine, as described herein, between about 0.01 wt. % and about 50 wt. % of an anti-inflammatory agent, as described herein, and between about 0.01 wt. % and about 50 wt. % of an amphiphilic agent, as described herein. In certain embodiments, a composition, as described herein, may comprise between about 50 wt. % and about 80 wt. % of beta-alanine, as described herein, between about 0.01 wt. % and about 10 wt. % of an anti-inflammatory agent, as described herein, and between about 0.1 wt. % and about 10 wt. % of an amphiphilic agent, as described herein. In certain embodiments, a composition, as described herein, can comprise between about 70 wt. % and about 80 wt. % of beta-alanine, as described herein, between about 0.1 wt. % and about 4 wt. % of an anti-inflammatory agent, as described herein, and between about 1 wt. % and about 4 wt. % of an amphiphilic agent, as described herein. In certain embodiments, a composition, as described herein, can comprise between about 70 wt. % and about 73 wt. % of beta-alanine, as described herein, between about 0.5 wt. % and about 1 wt. % of an anti-inflammatory agent, as described herein, and between about 1 wt. % and about 4 wt. % of an amphiphilic agent, as described herein.

In certain embodiments, a composition, as described herein, can be coated with a coating agent. Any suitable coating technique may be used, including spray coating, sugar coating, enteric coating, film coating, and the like. In certain embodiments, a coated composition, as described herein, may comprise between about 0.1 wt. % and about 50 wt. %, or between about 0.1 wt. % and about 20 wt. %, or between about 1 wt. % and about 10 wt. %, or between about 2 wt. % and about 5 wt. %, or between about 3 wt. % and about 4 wt. % of a coating agent, and ranges therebetween.

In some embodiments, a coating agent, as described herein, may be a particulate. Suitable coating agents falling within the scope of disclosure include, but are not limited to lactose, cellulose, microcrystalline cellulose, modified food starches, gum Arabic, maltodextrin, dextrose, xanthan gum, guar gum, carboxymethylcellulose, cellulose gel, cellulose gum, sodium caseinate, carrageenan, glucomannan, psyllium, gum acacia, polyethylene glycol, polyvinylpyrrolidone, hydroxypropyl methylcellulose, sodium carboxymethylcellulose, and combinations thereof. In some embodiments, a modified food starch, as described herein, is a starch that is chemically modified by esterification with a C₃-C₂₂ fatty acid chain. In some embodiments, a modified food starch, as described herein, can be a modified corn, potato, wheat, rice, tapioca, sago, oat, barley, amaranth, waxy maize, cassava, waxy barley, waxy rice, glutinous rice, or sweet rice starch. Exemplary modified food starches include, but are not limited to octenyl succinate starches, such as sodium octenyl succinate starch, calcium octenyl succinate starch, and aluminum octenyl succinate starch. A modified food starch, as described herein, can be a modified waxy maize starch, such as a sodium octenyl succinate waxy maize starch. In some embodiments, a modified food starch, as described herein, may be Capsul® (Starch sodium octenyl succinate).

In certain embodiments, a composition, as described herein, may further comprise a stimulant. A stimulant, as described herein, is a chemical that increases brain and nervous system activity, and increases the circulation of chemicals such as cortisol and adrenaline in the body. A stimulant, as described herein, can comprise caffeine, paraxanthine, methylhexanamine, salt thereof, or any combinations thereof. Caffeine can be from a natural source or synthesized. Examples of the natural source can include a green tea extract, a guarana extract, a coffee extract, or a yerba mate extract, lemon argentina, copaiba, orange sweet oil, grapefruit pink, or any combinations thereof. A stimulant, as described herein, can also comprise one or more of acetyl-L-carnitine/L-carnitine, theacrine, arginine, biotin, betel nut, B-vitamins, cacao, cayenne, chia seeds, chlorella, chlorophyll, chromium picolinate, coconut oil, coenzyme Q-10, cordyceps, damiana, dehydroepiandrosterone (DHEA), eluthero, ephedra, Fo-Ti (Polygonum multiflorum), one or more amino acids, Ginkgo biloba, red ginseng, Asian ginseng, Gotu kola/kula, grape seed, holy basil extract, hoodia, Indian ginseng, iodine, L-Theanine, L-Tyrosine, maca, Mucuna pruriens, niacin, Rhodiola rosea, sage, selenium, St. John's Wort, taurine, tea (Cammillia sinensis), Theobroma cacao, tobacco, xanthine, yohimbe, any extract of the foregoing, or a ground powder of any of the foregoing, and the like. An extract can refer to an active ingredient obtained from plants by physical (e.g., pressing) or chemical process (e.g., solvent extraction, distillation, and sublimation). Extracts can be substantially free of solvent, i.e., resulting in an active ingredient in a concentrated form. A ground powder refers to fine, dry particles produced by the grinding, crushing, or disintegration of a solid substance, and the like. In certain embodiments, a composition, as described herein, may comprise between about 0.01 wt. % and about 50 wt. %, or between about 0.05 wt. % and about 20 wt. %, or between about 0.1 wt. % and about 10 wt. %, or between about 0.1 wt. % and about 1 wt. %, or between about 0.1 wt. % and about 0.2 wt. % of a stimulant, and ranges therebetween.

In certain embodiments, a composition described herein comprises an effective amount of beta-alanine, an effective amount of an anti-inflammatory agent, and an effective amount of a stimulant. In certain embodiments, a composition can comprise at least 1 wt. % of beta-alanine, at most 99 wt. % of an anti-inflammatory agent, and at most about 99 wt. % of a stimulant. Certain compositions can comprise about 10 wt. %-99 wt. % of beta-alanine, about 0.01 wt. %-50 wt. % of an anti-inflammatory agent, and about 0.01 wt. %-50 wt. % of a stimulant. In certain embodiments, a composition comprises about 50 wt. %-80 wt. % of beta-alanine, 0.01 wt. %-10 wt. % of an anti-inflammatory agent, and 0.01 wt. %-10 wt. % of a stimulant. In certain embodiments, a composition comprises about 70 wt. %-80 wt. % of beta-alanine, about 0.1 wt. %-4 wt. % of an anti-inflammatory agent, and about 0.1 wt. %-1 wt. % of a stimulant. In certain embodiments, a composition comprises about 70 wt. %-75 wt. % of beta-alanine, about 0.5 wt. %-4 wt. % of an anti-inflammatory agent, and 0.1 wt. %-0.2 wt. % of a stimulant. In certain embodiments, a composition comprises about 70 wt. %-75 wt. % of beta-alanine, about 0.5 wt. %-3.5 wt. % of an anti-inflammatory agent, and about 0.17 wt. % of a stimulant. In certain embodiments, a composition comprises about 70 wt. %-73 wt. % of beta-alanine, about 0.5 wt. %-1.0 wt. % of an anti-inflammatory agent, and about 0.17 wt. % of a stimulant.

In certain embodiments, the amount of beta-alanine and the amount of an anti-inflammatory agent in a composition are present in a weight ratio ranging from 10:1 to 110:1 and the amount of an anti-inflammatory agent and the amount of a stimulant are present in a weight ratio ranging from 20:1 to 1:10. In certain embodiments, the amount of beta-alanine and the amount of an anti-inflammatory agent in a composition are present in a weight ratio ranging from 20:1 to 110:1 and the amount of an anti-inflammatory agent and the amount of a stimulant are present in a weight ratio ranging from 20:1 to 4:1.

In certain embodiments, a composition described herein comprises an effective amount of beta-alanine, an effective amount of an amphiphilic agent, and an effective amount of a stimulant. In certain embodiments, a composition described herein can comprise at least 1 wt. % of beta-alanine, at most 99 wt. % of an amphiphilic agent, and at most 99 wt. % of a stimulant. Certain compositions can comprise about 10 wt. %-99 wt. % of beta-alanine, about 0.01 wt. %-50 wt. % of an amphiphilic agent, and about 0.01 wt. %-50 wt. % of a stimulant. In certain embodiments, a composition as described herein comprises about 50 wt. %-80 wt. % of beta-alanine, 0.01 wt. %-10 wt. % of an amphiphilic agent, and 0.01 wt. %-10 wt. % of a stimulant. In certain embodiments, a composition as described herein comprises about 70 wt. %-80 wt. % of beta-alanine, about 1 wt. %-10 wt. % of an amphiphilic agent, and about 0.1 wt. %-1 wt. % of a stimulant. In certain embodiments, a composition as described herein comprises about 70 wt. %-75 wt. % of beta-alanine, about 2 wt. %-9 wt. % of an amphiphilic agent, and 0.1 wt. %-0.2 wt. % of a stimulant. In certain embodiments, a composition as described herein comprises about 70 wt % of beta-alanine, about 3 wt. % of an amphiphilic agent, and about 0.17 wt. % of a stimulant.

In certain embodiments, the amount of beta-alanine and the amount of an amphiphilic agent in a composition described herein can be present in a weight ratio ranging from 50:1 to 1:50 and the amount of an amphiphilic agent and the amount of a stimulant are present in a weight ratio ranging from 25:1 to 1:25. In certain embodiments, the amount of beta-alanine and the amount of an amphiphilic agent in a composition as described herein are present in a weight ratio ranging from 25:1 to 10:1 and the amount of an amphiphilic agent and the amount of a stimulant are present in a weight ratio ranging from 25:1 to 15:1.

The amount of beta-alanine in a composition, as described herein, may be about 0.01 g, 0.02 g, 0.03 g, 0.04 g, 0.05 g, 0.06 g, 0.07 g, 0.08 g, 0.09 g, 0.1 g, 0.15 g, 0.16 g, 0.17 g, 0.18 g, 0.19 g, 0.2 g, 0.21 g, 0.22 g, 0.23 g, 0.24 g, 0.25 g, 0.26 g, 0.27 g, 0.28 g, 0.29 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1.0 g, 1.1 g, 1.2 g, 1.3 g, 1.4 g, 1.5 g, 1.6 g, 1.7 g, 1.8 g, 1.9 g, 2.0 g, 2.1 g, 2.2 g, 2.3 g, 2.4 g, 2.5 g, 2.6 g, 2.7 g, 2.8 g, 2.9 g, 3.0 g, 3.1 g, 3.2 g, 3.3 g, 3.4 g, 3.5 g, 3.6 g, 3.7 g, 3.8 g, 3.9 g, 4.0 g, 4.1 g, 4.2 g, 4.3 g, 4.4 g, 4.5 g, 4.6 g, 4.7 g, 4.8 g, 4.9 g, 5.0 g, 5.1 g, 5.2 g, 5.3 g, 5.4 g, 5.5 g, 5.6 g, 5.7 g, 5.8 g, 5.9 g, 6.0 g, 6.1 g, 6.2 g, 6.3 g, 6.4 g, 6.5 g, 6.75 g, 7.0 g, 7.25 g, 7.5 g, 7.75 g, 8.0 g, 8.25 g, 8.5 g, 8.75 g, 9.0 g, 8.25 g, 9.5 g, 9.75 g, 10 g, 20 g, 30 g, 40 g, 50 g, 60 g, 70 g, 80 g, 90 g, 100 g, 110 g, 120 g, 130 g, 140 g, 150 g, 160 g, 170 g, 180 g, 190 g, 200 g, 500 g, 1000 g, 1500 g, 1600 g, 2000 g, 2500 g, 3000 g, 3200 g, 4000 g, 5000 g, 6000 g, 6400 g or more or any amount therebetween.

The amount of beta-alanine can be provided in an effective amount. As set forth herein, and as would be immediately envisaged by the skilled artisan in view of the disclosure contained herein, an effective amount can be an amount capable of achieving the desired outcome while also being provided at a safe amount.

The amount of an anti-inflammatory agent in a composition, as described herein, may be about 0 μg, 1 μg, 2 μg, 3 μg, 4 μg, 5 μg 6 μg 7 μg, 8 μg 9 μg 10 μg, 11 μg, 12 μg, 13 μg, 14 μg, 15 μg, 16 μg, 17 μg, 18 μg, 19 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 125 μg, 150 μg, 175 μg, 200 μg, 225 μg, 250 μg, 275 μg, 300 μg, 325 μg, 350 μg, 375 μg, 400 μg, 425 μg, 450 μg, 475 μg, 500 μg, 525 μg, 575 μg, 600 μg, 625 μg, 650 μg, 675 μg, 700 μg, 725 μg, 750 μg, 775 μg, 800 μg, 825 μg, 850 μg, 875 μg, 900 μg, 925 μg, 950 μg, 975 μg, 1000 μg, 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 11 mg, 12 mg, 13 mg, 14 mg, 15 mg, 16 mg, 17 mg, 18 mg, 19 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1.25 g, 1.5 g, 1.75 g, 2.0 g, 2.25 g, 2.5 g, 2.75 g, 3.0 g, 3.25 g, 3.5 g, 3.5 g, 3.75 g, 4.0 g, 4.25 g, 4.5 g, 4.75 g, 5.0 g, 5.25 g, 5.5 g, 5.75 g, 6.0 g, 6.25 g, 6.5 g, 6.75 g, 7.0 g, 7.25 g, 7.5 g, 7.75 g, 8.0 g, 8.25 g, 8.5 g, 8.75 g, 9.0 g, 8.25 g, 9.5 g, 9.75 g, 10 g, or more, or any amount therebetween.

The amount of an amphiphilic agent in a composition, as disclosed herein, may be about 0 μg, 1 μg, 2 μg, 3 μg, 4 μg, 5 μg, 6 μg, 7 μg, 8 μg, 9 μg, 10 μg, 15 μg, 20 μg, 25 μg, 30 μg, 35 μg, 40 g, 45 μg, 50 μg, 55 μg, 60 μg, 65 μg, 70 μg, 75 μg, 80 μg, 85 μg, 90 μg, 95 μg, 100 μg, 125 μg, 150 g, 175 μg, 200 μg, 225 μg, 250 μg, 275 μg, 300 μg, 325 μg, 350 μg, 375 μg, 400 μg, 425 μg, 450 μg, 475 μg, 500 μg, 525 μg, 575 μg, 600 μg, 625 μg, 650 μg, 675 μg, 700 μg, 725 μg, 750 μg, 775 μg, 800 μg, 825 μg, 850 μg, 875 μg, 900 μg, 925 μg, 950 μg, 975 μg, 1000 μg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1.25 g, 1.5 g, 1.75 g, 2.0 g, 2.25 g, 2.5 g, 2.75 g, 3.0 g, 3.25 g, 3.5 g, 3.5 g, 3.75 g, 4.0 g, 4.25 g, 4.5 g, 4.75 g, 5.0 g, 5.25 g, 5.5 g, 5.75 g, 6.0 g, 6.25 g, 6.5 g, 6.75 g, 7.0 g, 7.25 g, 7.5 g, 7.75 g, 8.0 g, 8.25 g, 8.5 g, 8.75 g, 9.0 g, 8.25 g, 9.5 g, 9.75 g, 10 g, or more, or any amount therebetween.

The amount of a stimulant in a composition as described herein may be about 0 μg, 1 μg, 2 μg, 3 μg, 4 μg, 5 μg, 6 μg, 7 μg, 8 μg, 9 μg, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 125 μg, 150 μg, 175 g, 200 μg, 225 μg, 250 μg, 275 μg, 300 μg, 325 μg, 350 μg, 375 μg, 400 μg, 425 μg, 450 μg, 475 μg, 500 μg, 525 μg, 575 μg, 600 μg, 625 μg, 650 μg, 675 μg, 700 μg, 725 μg, 750 μg, 775 μg, 800 μg, 825 μg, 850 μg, 875 μg, 900 μg, 925 μg, 950 μg, 975 μg, 1000 μg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1.25 g, 1.5 g, 1.75 g, 2.0 g, 2.25 g, 2.5 g, 2.75 g, 3.0 g, 3.25 g, 3.5 g, 3.5 g, 3.75 g, 4.0 g, 4.25 g, 4.5 g, 4.75 g, 5.0 g, 5.25 g, 5.5 g, 5.75 g, 6.0 g, 6.25 g, 6.5 g, 6.75 g, 7.0 g, 7.25 g, 7.5 g, 7.75 g, 8.0 g, 8.25 g, 8.5 g, 8.75 g, 9.0 g, 8.25 g, 9.5 g, 9.75 g, 10 g, or more, or any amount therebetween.

Compositions described herein may contain any number of additional components, including a diluent, a lubricant, a binder, disintegrating agents, coloring agents, wetting agents, preservatives, stabilizers, buffer substances, antioxidants, emulsifying agents, suspending agents, solubilizing agents, pH buffering agents, anticaking agents, sweetening agents, flavor agents and the like. Solvents including water, glycerin, sorbitol, ethyl alcohol, syrup and the like can be used in the compositions described herein. Examples of diluents include, but are not limited to, lactose, sucrose, dicalcium phosphate, and carboxy methylcellulose. Examples of lubricants include, but are not limited, to magnesium stearate, calcium stearate and talc. Examples of binders include, but are not limited to, starch, natural gums (e.g., gum acacia, arabic gum, xanthan gum), gelatin, glucose, molasses, polyvinylpyrrolidone, celluloses and derivatives thereof, povidone, and crospovidone. Examples of coloring agents include, but are not limited to, any of the approved certified water-soluble FD&C dyes. Examples of preservatives include, but are not limited to, glycerin, methyl and propylparaben, benzoic add, sodium benzoate and alcohol. Examples of non-aqueous liquids utilized in emulsions include, but are not limited to, mineral oil and cottonseed oil. Examples of emulsifying agents include, but are not limited to, gelatin, acacia, tragacanth, bentonite, and surfactants such as polyoxyethylene sorbitan monooleate. Examples of suspending agents include, but are not limited to, sodium carboxymethylcellulose, pectin, tragacanth, Veegum and acacia. Examples of wetting agents include, but are not limited to, propylene glycol monostearate, sorbitan monooleate, diethylene glycol monolaurate and polyoxyethylene lauryl ether. Examples of pH buffering agents include, but are not limited to, acetate, sodium citrate, cyclodextrine derivatives, sorbitan monolaurate, triethanolamine sodium acetate, and triethanolamine oleate. Examples of anticaking agents include, but are not limited to calcium silicate, stearates of calcium and magnesium, silica and various silicates, talc, flour, starch, and ferrocyanides. Examples of sweetening agents include, but are not limited to, glucose, sucrose, sucralose, syrups, lactitol, maltitol, xylitol, alcohol sugars, sorbitol, glycerin and artificial sweetening agents such as saccharin, aspartame, and acesulfame potassium. Flavor agents can include natural substances (e.g., wood particles, nut particles, fruit rinds, honey, etc), essential oil, oleoresin, essence or extractive, protein hydrolysate, distillate, or any product of roasting, heating or enzymolysis, which contains the flavoring constituents derived from a spice, fruit or fruit juice, vegetable or vegetable juice, edible yeast, herb, bark, bud, root, leaf or similar plant material, meat, seafood, poultry, eggs, dairy products, or fermentation products thereof, and the like, whose significant function in food is flavoring rather than nutritional. Flavor agents can also include artificial substances and/or flavorings. Examples of flavoring agents are natural or synthetic vanilla flavor, natural or synthetic chocolate flavor, natural or synthetic coffee flavor, natural or synthetic cocoa flavor, citric acid, licorice extract, cinnamon oil, eucalyptus oil, peppermint oil, clove oil, bay oil, thyme oil, lemon oil, lime oil, orange oil, grape oil, grapefruit oil, apple essence, pear essence, peach essence, strawberry essence, watermelon essence, raspberry essence, cherry essence, plum essence, pineapple essence, apricot essence and the like.

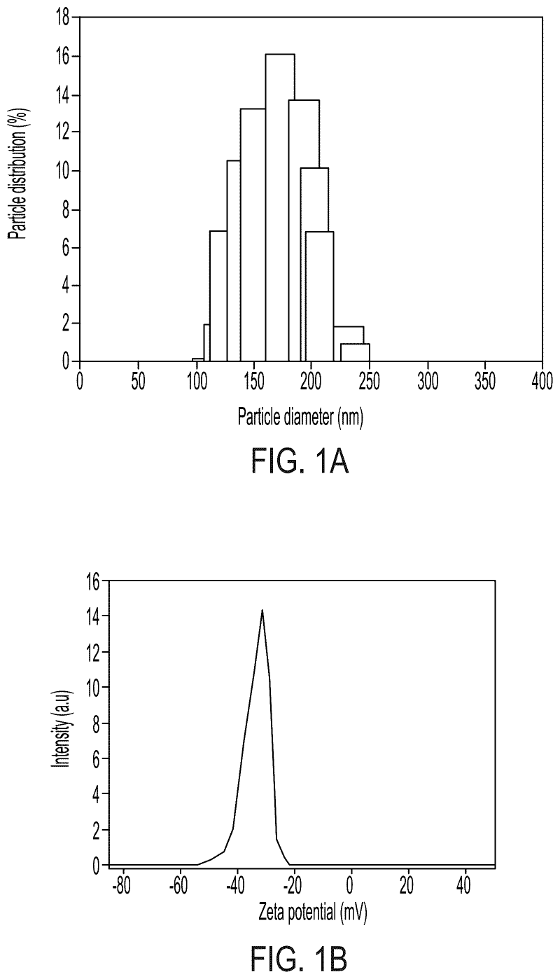

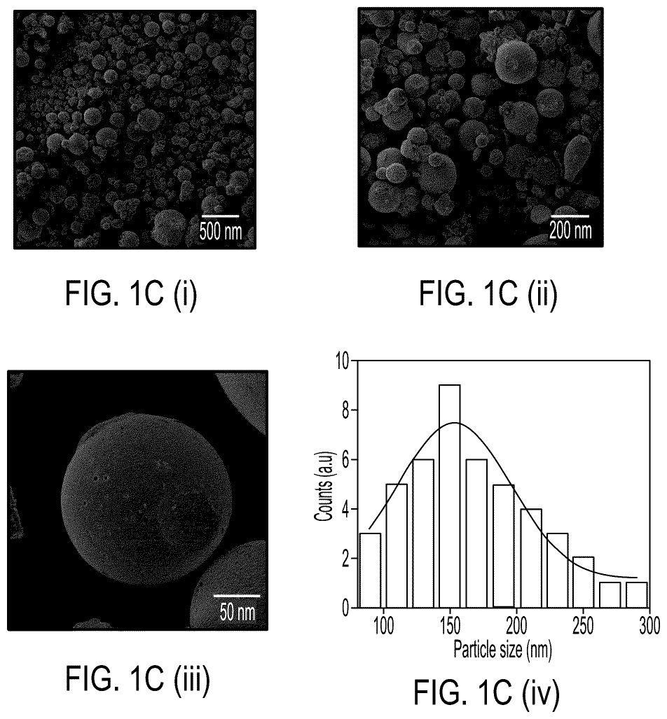

Compositions described herein possess unexpectedly superior particle uniformity and distributions, which confer improved aqueous solubility. For example, as shown in FIGS. 1A-1C, compositions described herein can comprise particles having an average particle size of about 160-180 nm, about 165-175 nm, about 170-175 nm, about 171.3±4.6 nm, and ranges therebetween by the Dynamic Light Scattering (DLS) method and having an average particle size of about 140-180 nm, about 145-165 nm, about 150-160 nm, about 150 nm, and ranges therebetween by Scanning Electron Microscope (SEM). Compositions described herein can have negative zeta potential greater than about −20 mV, greater than about −25 mV, greater than about −30 mV, or about −31 mV. As used herein, the higher of an absolute value, the greater of negative zeta potential. For example, negative zeta potential of −30 my is greater than negative zeta potential of −20 mV. Given the particle size distribution, improved particle size uniformity, and negative zeta potential, the compositions described herein exhibit unexpectedly improved stability (which provides for improved stability and shelf-life of dietary supplements described herein) and aqueous solubility (which provides their uses as ready-to-make products such as ready-to-make dietary supplements described herein). As discussed herein, compositions described herein can be formulated as ready-to-make products (e.g., a powder, a capsule, and a tablet) that can be mixed with water before consumption.

In certain embodiments, a composition can be in a sustained-release formulation. Sustained release refers to any one of extended-release dosage forms. The following terms may be considered to be substantially equivalent to sustained release, for the purposes of the present disclosure: continuous release, controlled release, delayed release, depot, gradual release, long term release, programmed release, prolonged release, programmed release, proportionate release, protracted release, repository, retarded release, slow release, spaced release, time coat, time release, delayed action, extended action, layered time action, long acting, prolonged action, sustained action medications, extended release, and release in terms of pH level in the gut and intestine.

In certain embodiments, the sustained-release formulation comprises a physical system. Examples of the physical system include, but are not limited to, reservoir systems with rate-controlling membranes such as encapsulation (including micro-encapsulation and macro-encapsulation) and membrane systems; reservoir systems without rate-controlling membranes such as hollow fibers, ultra macroporous cellulose triacetate, and porous polymeric substrates and foams; monolithic systems including systems physically dissolved in non-porous, polymeric, or elastomeric matrices (e.g., non-erodible, erodible, environmental agent ingression, and degradable) and materials physically dispersed in non-porous, polymeric, or elastomeric matrices (e.g., non-erodible, erodible, environmental agent ingression, and degradable); laminated structures including reservoir layers chemically similar or dissimilar to outer control layers; other physical methods such as osmotic pumps or adsorption onto ion-exchange resins and the like.

In certain embodiments, the sustained-release formulation comprises a chemical system. Examples of the chemical system include, but are not limited to, chemical erosion of polymer matrices (e.g., heterogeneous or homogeneous erosion), biological erosion of a polymer matrix (e.g., heterogeneous or homogeneous), hydrogels and the like.

In certain embodiments, the sustained-release formulation is a microencapsulated powder; a osmotic pressure-controlled gastrointestinal delivery system; a hydrodynamic pressure-controlled gastrointestinal delivery system; a membrane permeation-controlled gastrointestinal delivery system, including microporous membrane permeation-controlled gastrointestinal delivery devices; a gel diffusion controlled gastrointestinal delivery system; and an ion-exchange-controlled gastrointestinal delivery system, including cationic and anionic drugs and the like. In one embodiment, the sustained-release formulation can be an oil-microencapsulated sustained release powder dosage form that can be mixed with liquid and consumed as a drink mix beverage.

In certain embodiments, the sustained-release formulation can be obtained by using sufficient amount of the conventional pharmaceutical binders, excipients and additives as sustained-release polymers to produce a sustained-release effect. These include, but are not limited to, gelatin, natural sugars (e.g., raw sugar and lactose), lecithin, mucilage, plant gums, pectins, pectin derivatives, algal polysaccharides, glucomannan, agar, lignin, guar gum, locust bean gum, acacia gum, xanthan gum, carrageenan gum, karaya gum, tragacanth gum, ghatti gum, starches (e.g., corn starch and amylose), dextran, polyvinyl pyrrolidone, polyvinyl acetate, gum arabic, alginic acid, tylose, talcum, lycopodium, silica gel (e.g., colloidal), cellulose, cellulose derivatives (e.g., cellulose ethers, methoxypropyl cellulose, methyl cellulose, hydroxypropyl methyl cellulose, hydroxypropyl methyl cellulose phthalate, cross-linked sodium carboxymethylcellulose, cross-linked hydroxypropylcellulose, high-molecular weight hydroxymethylpropylcellulose, carboxymethyl-cellulose, low-molecular weight hydroxypropylmethylcellulose medium-viscosity hydroxypropylmethylcellulose hydroxyethylcellulose, hydroxypropylcellulose, hydroxypropylmethylcellulose, sodium carboxymethylcellulose, alkyl celluloses, ethyl cellulose, cellulose acetate, cellulose propionate, cellulose acetatepropionate, cellulose acetate butyrate, cellulose triacetate, methyl cellulose, hydroxypropyl cellulose, and hydroxypropylmethyl cellulose), fatty acids including saturated fatty acids (e.g., stearates such as magnesium stearate), glycerol esters and polyglycerol esters of saturated fatty acids and their mixtures, magnesium salts of fatty acids, calcium salts of fatty acids, aluminum salts of fatty acids, polycarboxylic acids, emulsifiers, oils and fats including vegetable oils (e.g., peanut oil, castor oil, olive oil, sesame oil, cottonseed oil, corn oil, wheat germ oil, sunflower seed oil, and cod fiver oil), hydrogenated vegetable oils suitable for microencapsulation, pharmaceutically acceptable monovalent or multivalent alcohols and polyglycols such as polyethylene glycol and derivatives thereof, esters of aliphatic saturated or unsaturated fatty acids with monovalent aliphatic alcohols, multivalent alcohols (e.g., glycols, glycerol, diethylene glycol, pentacrythritol, sorbitol, and mannitol), esterified monovalent aliphatic alcohols, esterified multivalent alcohols, esters of citric acid with primary alcohols, acetic acid, urea, benzyl benzoate, dioxolanes, glycerofonnals, tetrahydrofurfuryl alcohol, polyglycol ethers, dimethylacetamide, lactamides, lactates, ethylcarbonates, silicones (e.g., medium-viscous polydimethyl siloxanes), calcium carbonate, sodium carbonate, calcium phosphate, sodium phosphate, magnesium carbonate and the like.

In certain embodiments, the sustained-release formulation can be obtained by encapsulating beta-alanine with one or more coating agents to create or enhance the sustained release features of the composition. For example, plasticizers that are useful as coating agents include, but are not limited to, citric and tartaric acid esters (e.g., acetyl-triethyl citrate, acetyl tributyl-citrate, tributyl-citrate, and triethyl-citrate); glycerol and glycerol esters (e.g., glyceroldiacetate, glyceroltriacetate, acetylated monoglycerides, and castor oil); phthalic acid esters (e.g., dibutyl-phthalate, diamyl-phthalate, diethyl-phthalate, dimethyl-phthalate, dipropyl-phthalate), di-(2-methoxy-or 2-ethoxyethyl)-phthalate, ethylphthalyl glycolate, butylphthalylethyl glycolate, and butylglycolate; alcohols (e.g., propylene glycol, and polyethylene glycol of various chain lengths); adipates (e.g., diethyladipate, di (2-methoxy)-adipate, and di(2-ethoxyethyl)-adipate); benzophenone; diethyl-sebacate; diburylsebacate; dibutylsuccinate; dibutyltartrate; diethylene glycol dipropionate; ethyleneglycol diacetate; ethyleneglycol dibutyrate; ethyleneglycol dipropionate; tributyl phosphate; tributyrin; polyethylene glycol sorbitan monooleate; polysorbates such as Polysorbar 50; sorbitan monooleate and the like.

Certain embodiments comprise a beta-alanine composition comprising an encapsulated beta-alanine composition and an unencapsulated beta-alanine composition as described herein. As described herein, an encapsulated beta-alanine composition can refer to beta-alanine that is substantially present as encapsulated within an amphiphilic agent. As described herein, an unencapsulated beta-alanine composition can refer to beta-alanine that is not encapsulated with an amphiphilic agent. It is believed that a beta-alanine composition comprising an encapsulated beta-alanine composition and an unencapsulated beta-alanine composition has a dual effect; for example, an encapsulated beta-alanine composition can increase the amount of beta-alanine in cells/tissues in a short period of time and thus provides an instant effect and an unencapsulated beta-alanine composition can provide an additional effect, for example, a constant amount of beta-alanine in the cells/tissues and thus provides a long-lasting effect, for example, when provided as in an unencapsulated sustained release formulation. As described herein, both an encapsulated beta-alanine composition and an unencapsulated beta-alanine composition can be in sustained-release formulations. In certain embodiments, a beta-alanine composition comprises an encapsulated beta-alanine composition in a conventional-release formulation and an unencapsulated beta-alanine composition in a sustained-release formulation. In certain embodiments, a beta-alanine composition comprises an encapsulated beta-alanine composition in a sustained-release formulation and an unencapsulated beta-alanine composition in a conventional-release formulation. In certain embodiments, a beta-alanine composition comprises an encapsulated beta-alanine composition and an unencapsulated beta-alanine composition both in sustained-release formulations. In certain embodiments, a beta-alanine composition comprises an encapsulated beta-alanine composition and an unencapsulated beta-alanine composition both in conventional-release formulations. Conventional release refers to a beta-alanine composition that is not formulated as sustained release, as described herein and can refer to compositions wherein no deliberate effort is made to modify the beta-alanine release rate.

In some embodiments, a composition described herein can comprise an effective amount of an encapsulated beta-alanine and an effective amount of an unencapsulated beta-alanine. In certain embodiments, a composition comprises at least 1 wt. % an encapsulated beta-alanine and at most 99 wt. % an unencapsulated beta-alanine. In certain embodiments, a composition comprises at most 99 wt. % an encapsulated beta-alanine and at least 1 wt. % an unencapsulated beta-alanine. In certain embodiments, the amount of an encapsulated beta-alanine and the amount of an unencapsulated beta-alanine in a composition can be present in a weight ratio ranging from 99:1 to 1:99. In certain embodiments, the amount of an encapsulated beta-alanine and the amount of an unencapsulated beta-alanine in a composition can be present in a weight ratio ranging from 9:1 to 1:9. In certain embodiments, the amount of an encapsulated beta-alanine and the amount of an unencapsulated beta-alanine in a composition can be present in a weight ratio ranging from 4:1 to 1:4. In certain embodiments, the amount of an encapsulated beta-alanine and the amount of an unencapsulated beta-alanine in a composition can be present in a weight ratio ranging from 7:3 to 3:7. In certain embodiments, the amount of an encapsulated beta-alanine and the amount of an unencapsulated beta-alanine in a composition can be present in a weight ratio ranging from 3:2 to 2:3. In certain embodiments, the amount of an encapsulated beta-alanine and the amount of an unencapsulated beta-alanine in a composition can be present in a weight ratio of 1:1.

The amount of an unencapsulated beta-alanine and the amount of an encapsulated beta-alanine in a composition independently may be about 0.005 g, 0.01 g, 0.02 g, 0.03 g, 0.04 g, 0.05 g, 0.06 g, 0.07 g, 0.08 g, 0.09 g, 0.1 g, 0.15 g, 0.16 g, 0.17 g, 0.18 g, 0.19 g, 0.2 g, 0.21 g, 0.22 g, 0.23 g, 0.24 g, 0.25 g, 0.26 g, 0.27 g, 0.28 g, 0.29 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1.0 g, 1.1 g, 1.2 g, 1.3 g, 1.4 g, 1.5 g, 1.6 g, 1.7 g, 1.8 g, 1.9 g, 2.0 g, 2.1 g, 2.2 g, 2.3 g, 2.4 g, 2.5 g, 2.6 g, 2.7 g, 2.8 g, 2.9 g, 3.0 g, 3.1 g, 3.2 g, 3.3 g, 3.4 g, 3.5 g, 3.6 g, 3.7 g, 3.8 g, 3.9 g, 4.0 g, 4.1 g, 4.2 g, 4.3 g, 4.4 g, 4.5 g, 4.6 g, 4.7 g, 4.8 g, 4.9 g, 5.0 g, 5.1 g, 5.2 g, 5.3 g, 5.4 g, 5.5 g, 5.6 g, 5.7 g, 5.8 g, 5.9 g, 6.0 g, 6.1 g, 6.2 g, 6.3 g, 6.4 g, 6.5 g, 6.75 g, 7.0 g, 7.25 g, 7.5 g, 7.75 g, 8.0 g, 8.25 g, 8.5 g, 8.75 g, 9.0 g, 8.25 g, 9.5 g, 9.75 g, 10 g, 20 g, 30 g, 40 g, 50 g, 60 g, 70 g, 80 g, 90 g, 100 g, 110 g, 120 g, 130 g, 140 g, 150 g, 160 g, 170 g, 180 g, 190 g, 200 g, 500 g, 1000 g, 1500 g, 1600 g, 2000 g, 2500 g, 3000 g, 3200 g, 4000 g, 5000 g, 6000 g, 6400 g or more or any amount therebetween.

In certain embodiments, a composition as described herein may be formulated as a pharmaceutical composition for humans and animals. The pharmaceutical composition may comprise a pharmaceutically acceptable vehicle, carrier, or diluent. Pharmaceutically acceptable vehicle, carriers and diluent are known in the art and their application would be immediately envisaged by the skilled artisan in view of the disclosure contained herein, and their selections usually are dependent upon the intended route of delivery. The pharmaceutical composition can be liquids, semi-liquids, capsules, tablets, pills, powders, granules, sterile parenteral solutions or suspensions, oral solutions or suspensions, oil water emulsions, implants and microencapsulated delivery systems, and biodegradable, biocompatible polymers such as collagen, ethylene vinyl acetate, polyanhydrides, polyglycolic acid, polyorthoesters, and polylactic acid. Carriers for a liquid composition include, for example, water, saline, aqueous dextrose, glycerol, glycols, ethanol, and the like. Modes of delivery of the pharmaceutical compositions include oral delivery, mucosal delivery, nasal delivery, ocular delivery, transdermal delivery, parenteral delivery, vaginal delivery, rectal delivery, intrauterine delivery and the like.

In certain embodiments, a composition as described herein can be formulated as a dietary supplement for human and animals. Formulations of a dietary supplement can include, but is not limited to, tablets, capsules, granules, sachet, gummy, powders, gel, a beverage such as a sports drink, a soft drink (carbonated and non-carbonated), tea, iced tea, a fruit flavored drink, juice, and an energy drink, an energy bar, functional foods, medical foods, a food ingredient, or the like. Types of oral tablets include compressed, chewable lozenges and tablets which may be enteric coated, sugar coated, film coated or the like. Capsules can be hard gelatin capsules, soft gelatin capsules, or vegetarian capsules (such as hard HPMC capsules). By way of example, a composition described herein can be suspended in, for example, propylene carbonate, vegetable oils and/or triglycerides and encapsulated in a gelatin capsule. Granules and powders can be provided in a non-effervescent or an effervescent form with a combination of other ingredients known to those skilled in the art.

In certain embodiments, compositions described herein can be administered once a day, twice a day, three times a day, four times a day, five times a day, six times a day, seven times a day, eight times a day, nine times a day, ten times a day, or more times a day.

In certain embodiments, a dietary supplement contains about 0.01 g to about 200 g, about 0.02 g to about 10 g, about 0.03 g to about 6.4 g, about 0.04 g to about 3.2 g, about 0.05 g to about 2.24 g, about 0.1 g to about 1 g, about 0.2 g to 0.8 g, about 0.3 g to 0.6 g, about 0.2 g to 0.5 g, or about 0.2 g to 0.3 g of beta-alanine. In certain embodiments, a human dietary supplement contains about 0.1 g, 0.15 g, 0.2 g, 0.21 g, 0.22 g, 0.23 g, 0.24 g, 0.25 g, 0.26 g, 0.27 g, 0.28 g, 0.29 g, 0.3 g, 0.31 g, 0.32 g, 0.33 g, 0.34 g, 0.35 g, 0.36 g, 0.37 g, 0.38 g, 0.39 g, 0.4 g, 0.45 g, 0.5 g, 0.55 g, 0.6 g, 0.65 g, 0.7 g, 0.75 g, 0.8 g, 0.85 g, 0.9 g, 0.95 g, 1.0 g, 1.1 g, 1.2 g, 1.6 g, 2.24 g, 2.4 g, 3.2 g, 4.8 g, or 6.4 g of beta-alanine. In certain embodiments, a human dietary supplement contains about 0 mg to about 10 g, about 1 mg to about 1 g, about 2 mg to about 500 mg, about 3 mg to about 350 mg, about 4 mg to about 150 mg, about 5 mg to about 100 mg, about 6 mg to about 50 mg, about 7 mg to about 25 mg, about 2 mg to about 4 mg, or about 10 mg to about 20 mg of an anti-inflammatory agent. In certain embodiments, a human dietary supplement contains about 0 mg to about 10 g, about 0.1 mg to about 1 g, about 1 mg to about 500 mg, about 3 mg to about 250 mg, about 5 mg to about 150 mg, about 9 mg to about 100 mg, or about 10 mg to about 20 mg of an amphiphilic agent. In certain embodiments, a human dietary supplement contains about 0 mg to about 10 g, about 10 mg to about 1 g, about 20 mg to about 500 mg, about 30 mg to about 250 mg, about 40 mg to about 150 mg, about 50 mg to about 100 mg, or about 0.1 mg to about 1 mg of a stimulant. In certain embodiments, a human dietary supplement described herein is administered twice a day.

In certain embodiments, a dietary supplement described herein may include other ingredients such as amino acids, carbohydrates (e.g., starch and/or sugars, e.g., glucose, fructose, sucrose, and maltrose), insulin, insulin mimics, insulin-action modifier, glycine, fiber, fat, creatine, beta-alanylhistidine peptides (e.g. carnosine, anserine, and/or balenine), galactose, electrolytes, vitamins (e.g., vitamin E, vitamin C, thiamin, riboflavin, niacin, vitamin B6, folic acid, vitamin B12, biotin, and pantothenic acid), lipids, trace elements (e.g., calcium and potassium in the form of their gluconates, phosphates or hydrogen phosphates, and magnesium as the oxide or carbonate, chromium as chromium picolinate, selenium as sodium selenite or selenate, and zinc as zinc gluconate), colorings, natural health improving substances, probiotics, anti-oxidants (e.g., green tea polyphenols and grape seed extract), stabilizers, preservatives, alpha-lipoic acid, tocotrienols, N-acetylcysteine, co-enzyme Q-10, rosemary extracts, COX-1 type inhibitors (e.g., resveratrol), Ginkgo biloba, garlic extracts, buffers and the like.

A composition disclosed herein can be administered to a subject to prevent, treat, maintain healthy levels of, or alleviate declined brain functions, mood disorders, and mental fatigue. Brain functions include, but are not limited to, executive function, mental clarity, cognitive function, memory, attention, task-focus, concentration, alertness, and responses to everyday stress. Declined brain functions can be caused by aging, which include, but are not limited to, dementia, memory decline, and decline in cognitive abilities. Mood disorders refer to emotional state or a mood that is distorted or inconsistent with a subject's circumstances and interferes with a subject's ability to function. Examples of mood disorders include, but are not limited to, depression, anxiety, major depression, dysthymia (dysthymic disorder), bipolar disorder, mood disorder linked to another health condition, mood disorders due to aging (e.g., age-related depression and anxiety), and substance-induced mood disorder. In certain embodiments, a composition described herein can be administered to a subject to maintain healthy levels of brain functions and mood balance. In certain embodiments, a composition described herein can be administered to a subject to restore brain functions and mood balance to healthy levels. In certain embodiments, a composition described herein can be administered to a subject to improve executive function and mental clarity, reduce mental fatigue, improve cognitive function, prevent memory loss, improve memory, enhance mental acuity, improve attention and task focus, improve concentration and alertness, promote healthy responses to everyday stress, foster balanced mood, and maintain calmness under pressure. In certain embodiments, a composition described herein can be administered to a subject to prevent, treat, maintain healthy levels of, or alleviate declined brain functions (e.g., dementia, memory decline, and a decline in cognitive abilities), mood disorders (e.g., depression and anxiety), and mental fatigue caused by aging. In certain embodiments, a composition described herein can be administered to a subject to maintain healthy levels of brain functions and mood balance during aging. In certain embodiments, a composition described herein can be administered to a subject to restore brain functions and mood balance to healthy levels, improve executive function and mental clarity, reduce mental fatigue, improve cognitive function, prevent dementia, prevent memory loss, improve memory, improve attention and task focus, improve concentration and alertness, promote healthy responses to everyday stress, foster balanced mood, reduce anxiety, and maintain calmness under pressure.

A composition disclosed herein can be administered to a subject to prevent, treat or alleviate a weakened immune system. A composition described herein can also be administered to a subject to maintain a healthy immune system and/or restore weaken immune system to heathy levels. A weakened immune system can be caused by aging, among other factors. In certain embodiments, a composition described herein can be administered to a subject to maintain and support healthy immune function and structures. Healthy immune function refers to healthy immune response and everyday wellness. In certain embodiments, a composition described herein can be administered to a subject to prevent and/or alleviate oxidative damages in cells. In certain embodiments, a composition described herein can be administered to a subject to promote healthy aging. In certain embodiments, a composition described herein can be administered to a subject to attenuate the aging process. In certain embodiments, a composition described herein can be administered to a subject to slow down age-related changes, including, but are not limited to, memory decline, drier skin, a decline in cognitive abilities, age-related muscle atrophy, muscle wasting, sarcopenia, age-related bone changes (e.g., bone loss, bone brittleness, bone spurs, shorter trunk and spine, less pronounced foot arches, joint breakdown, and increased risk of fractures), cartilage wear and tear, depression, anxiety, dementia, increased risk of heart diseases, and increased risk of diabetes.

A composition disclosed herein can be administered to a subject to prevent, treat, or alleviate declined muscle strength and/or bone/joint dysfunctions, which may be caused by aging, among other factors. A composition disclosed herein can also be administered to a subject to maintain healthy muscle strength (such as a healthy level of muscle mass) and/or bone/joint functions and/or restore muscle strength and bone/joint functions to healthy levels. As muscle mass closely correlates to physical performance, a composition disclosed herein can be administered to a subject to maintain a healthy level of physical performance. Examples of declined muscle strength include, but are not limited to, muscle fatigue and loss of muscle mass. Examples of bone dysfunctions include, but are not limited to, low bone density, osteoporosis, osteogenesis imperfecta, Paget's disease of bone, cancer, infections, and other bone diseases caused by aging, poor nutrition, genetics, or problems with the rate of bone growth or rebuilding. A subject may have low bone mass at any age but not develop osteoporosis. Examples of joint dysfunctions include, but are not limited to, arthritis (e.g., osteoarthritis) and bursitis. In certain embodiments, a composition can be administered to a subject to increase muscle strength and/or working capacity, improve endurance, and/or delaying the onset of muscle fatigue during exercise or physical exertion, including everyday tasks that become more difficult with age along with high-intensity exercise. Work capacity refers to the ability to perform real physical work and can be assessed by aerobic capacity, endurance, energetic efficiency, voluntary activity, and work productivity. Endurance is defined as the maximum length of time a subject can sustain a given workload without rest. In certain embodiments, a composition can be administered to a subject to prevent bone loss or restore bone density to healthy levels. In certain embodiments, a composition can be administered to a subject to maintain healthy joints or restore joint to healthy levels. In certain embodiments, a composition described herein can be administered to a subject to maintain healthy levels of muscle mass, function, quality, strength, and working capacity during aging. In certain embodiments, a composition described herein can be administered to a subject to stimulate bone growth, maintain healthy levels of bone mass, or reduce bone loss. In certain embodiments, a composition described here can be administered to a subject to maintain healthy levels of bone mass during aging. In certain embodiments, a composition can be administered to a subject to maintain joint health during aging. In certain embodiments, a composition described herein can be administered to a subject to alleviate age-related muscle loss, bone density loss, and cartilage wear and tear. In certain embodiments, a composition can be administered to a subject age 30 or older to promote a healthy level of physical performance.

A composition disclosed herein can be administered to a subject to prevent and treat diabetes and cardiovascular diseases. In certain embodiments, a composition disclosed herein can be administered to an overweight and obese subject to improve the plasma lipidome. In certain embodiments, a composition disclosed herein can be administered to an overweight and obese subject to normalize the serum adipokine concentrations. In certain embodiments, a composition disclosed herein can be administered to a subject to maintain healthy blood sugar levels. In certain embodiments, a composition disclosed herein can be administered to a subject to improve contractility and cardiac function of heart and blood circulation. In certain embodiments, a composition disclosed herein can be administered to a subject to maintain healthy levels of contractility and cardiac function of heart and blood circulation.

Also described herein is a method of increasing athletic performance in a subject without causing paresthesia or with reduced paresthesia effect. The method comprises administering to the subject an effective amount of a composition described herein to safely and effectively increase the synthesis of carnosine in muscle, to attenuate metabolic acidosis and muscle fatigue during physical exercise, and to obviate the paresthesia sensation. Examples of increased athletic performance include improved stamina, speed, strength, power, endurance, flexibility, agility, and balance, focus coordination, reaction time, and fatigue recovery and the like.

A method of improving cognitive function in a subject is also provided herein. The method comprises administering to the subject an effective amount of a composition described herein to safely and effectively increase the concentration of carnosine in brain and to obviate the paresthesia sensation. Improved cognitive function includes improved attention span, memory, reaction time, problem-solving ability, and mental alertness. In certain embodiments, the subject is an adult age 30 or older. Subjects being administered compositions described herein for the purposes described herein can be over the age of 40, over the age of 50, over the age of 60, over the age of 65, over the age of 70, over the age of 80, and over the age of 90.

Methods of attenuating the aging process (i.e., anti-aging functions) and promoting healthy aging and general wellness (including preventing age-related muscle atrophy, and maintaining a healthy level of muscle mass and physical performance) are also provided herein. The methods each comprise administering to a subject an effective amount of a composition described herein to safely and effectively increase carnosine concentrations in tissues in the body (including skeletal muscle, heart muscle, brain, and cells). In certain embodiments, the subject is an adult age 30 or older. Subjects being administered compositions described herein for the purposes described herein can be over the age of 40, over the age of 50, over the age of 60, over the age of 65, over the age of 70, over the age of 80, and over the age of 90.

A composition can be administered in a single dose or multiple doses per day. For example, a composition can be administered one time to twenty-four times in a 24-hour period. In certain embodiments, a composition can be administered twice a day. In certain embodiments, the total dosage of beta-alanine in a composition described herein for a 24-hour period may be about 0.1 g, 0.2 g, 0.21 g, 0.22 g, 0.23 g, 0.24 g, 0.25 g, 0.26 g, 0.27 g, 0.28 g, 0.29 g, 0.3 g, 0.31 g, 0.32 g, 0.33 g, 0.34 g, 0.35 g, 0.36 g, 0.37 g, 0.38 g, 0.39 g, 0.4 g, 0.41 g, 0.42 g, 0.43 g, 0.44 g, 0.45 g, 0.46 g, 0.47 g, 0.48 g, 0.49 g, 0.5 g, 0.51 g, 0.52 g, 0.53 g, 0.54 g, 0.55 g, 0.56 g, 0.57 g, 0.58 g, 0.59 g, 0.6 g, 0.7 g, 0.8 g, 0.9 g, 1.0 g, 1.1 g, 1.2 g, 1.3 g, 1.4 g, 1.5 g, 1.6 g, 1.7 g, 1.8 g, 1.9 g, 2.0 g, 2.1 g, 2.2 g, 2.3 g, 2.4 g, 2.5 g, 2.6 g, 2.7 g, 2.8 g, 2.9 g, 3.0 g, 3.1 g, 3.2 g, 3.3 g, 3.4 g, 3.5 g, 3.6 g, 3.7 g, 3.8 g, 3.9 g, 4.0 g, 4.1 g, 4.2 g, 4.3 g, 4.4 g, 4.5 g, 4.6 g, 4.7 g, 4.8 g, 4.9 g, 5.0 g, 5.1 g, 5.2 g, 5.3 g, 5.4 g, 5.5 g, 5.6 g, 5.7 g, 5.8 g, 5.9 g, 6.0 g, 6.1 g, 6.2 g, 6.3 g, 6.4 g, 6.5 g, 6.75 g, 7.0 g, 7.25 g, 7.5 g, 7.75 g, 8.0 g, 8.25 g, 8.5 g, 8.75 g, 9.0 g, 8.25 g, 9.5 g, 9.75 g, 10 g, 20 g, 30 g, 40 g, 50 g, 60 g, 70 g, 80 g, 90 g, 100 g, 110 g, 120 g, 130 g, 140 g, 150 g, 160 g, 170 g, 180 g, 190 g, 200 g or more or any amount therebetween. In certain embodiments, a composition described herein contains about 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1.0 g, 1.1 g, 1.2 g, 1.6 g, 2.24 g, 2.4 g, 3.2 g, 4.8 g, or 6.4 g of beta-alanine per serving.

The total dosage of an anti-inflammatory agent in a composition described herein for a 24-hour period may be about 0 μg, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 125 μg, 150 μg, 175 μg, 200 μg, 225 μg, 250 μg, 275 μg, 300 μg, 325 μg, 350 μg, 375 μg, 400 μg, 425 μg, 450 μg, 475 μg, 500 μg, 525 μg, 575 μg, 600 μg, 625 μg, 650 μg, 675 μg, 700 μg, 725 μg, 750 μg, 775 μg, 800 μg, 825 μg, 850 μg, 875 μg, 900 μg, 925 μg, 950 μg, 975 μg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1.25 g, 1.5 g, 1.75 g, 2.0 g, 2.25 g, 2.5 g, 2.75 g, 3.0 g, 3.25 g, 3.5 g, 3.5 g, 3.75 g, 4.0 g, 4.25 g, 4.5 g, 4.75 g, 5.0 g, 5.25 g, 5.5 g, 5.75 g, 6.0 g, 6.25 g, 6.5 g, 6.75 g, 7.0 g, 7.25 g, 7.5 g, 7.75 g, 8.0 g, 8.25 g, 8.5 g, 8.75 g, 9.0 g, 8.25 g, 9.5 g, 9.75 g, 10 g, or more, or any amount therebetween.

The total dosage of an amphiphilic agent in a composition described herein for a 24-hour period may be about 0 μg, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 125 μg, 150 μg, 175 μg, 200 μg, 225 μg, 250 μg, 275 μg, 300 μg, 325 μg, 350 μg, 375 μg, 400 μg, 425 μg, 450 μg, 475 μg, 500 μg, 525 μg, 575 μg, 600 μg, 625 μg, 650 μg, 675 μg, 700 μg, 725 μg, 750 μg, 775 μg, 800 μg, 825 μg, 850 μg, 875 μg, 900 μg, 925 μg, 950 μg, 975 μg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1.25 g, 1.5 g, 1.75 g, 2.0 g, 2.25 g, 2.5 g, 2.75 g, 3.0 g, 3.25 g, 3.5 g, 3.5 g, 3.75 g, 4.0 g, 4.25 g, 4.5 g, 4.75 g, 5.0 g, 5.25 g, 5.5 g, 5.75 g, 6.0 g, 6.25 g, 6.5 g, 6.75 g, 7.0 g, 7.25 g, 7.5 g, 7.75 g, 8.0 g, 8.25 g, 8.5 g, 8.75 g, 9.0 g, 8.25 g, 9.5 g, 9.75 g, 10 g, or more, or any amount therebetween.

The total dosage of a stimulant in a composition described herein for a 24-hour period may be about 0 μg, 1 μg, 2 μg, 3 μg, 4 μg, 5 μg, 6 μg, 7 μg, 8 μg, 9 μg, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 125 μg, 150 ag, 175 μg, 200 ag, 225 μg, 250 μg, 275 μg, 300 ag, 325 μg, 350 μg, 375 ag, 400 ag, 425 μg, 450 μg, 475 μg, 500 μg, 525 μg, 575 μg, 600 μg, 625 μg, 650 μg, 675 μg, 700 μg, 725 μg, 750 μg, 775 μg, 800 μg, 825 μg, 850 μg, 875 μg, 900 μg, 925 μg, 950 μg, 975 μg, 1000 μg, 1.1 mg, 1.2 mg, 1.3 mg, 1.4 mg, 1.5 mg, 1.6 mg, 1.7 mg, 1.8 mg, 1.9 mg, 2 mg, 3 mg, 4 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1.25 g, 1.5 g, 1.75 g, 2.0 g, 2.25 g, 2.5 g, 2.75 g, 3.0 g, 3.25 g, 3.5 g, 3.5 g, 3.75 g, 4.0 g, 4.25 g, 4.5 g, 4.75 g, 5.0 g, 5.25 g, 5.5 g, 5.75 g, 6.0 g, 6.25 g, 6.5 g, 6.75 g, 7.0 g, 7.25 g, 7.5 g, 7.75 g, 8.0 g, 8.25 g, 8.5 g, 8.75 g, 9.0 g, 8.25 g, 9.5 g, 9.75 g, 10 g, or more, or any amount therebetween. In certain embodiments, a composition described herein contains about 0.01 g to about 200 g, about 0.02 g to about 10 g, about 0.03 g to about 6.4 g, about 0.04 g to about 3.2 g, about 0.05 g to about 2.24 g, about 0.1 g to about 1 g, or about 0.2 g to 0.8 g of beta-alanine, or more and values therebetween, administered in a 24-hour period. In certain embodiments, a composition described herein contains about 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1.0 g, 1.1 g, 1.2 g, 1.6 g, 2.24 g, 2.4 g, 3.2 g, 4.8 g, or 6.4 g, or more and values therebetween, of beta-alanine administered in a 24-hour period.

A composition described herein for improving athletic performance can be given on a daily basis over a period of about 1 day to about four or more weeks. In certain embodiments, a composition described herein can be administered for about 28 days. In certain embodiments, a composition described herein can be administered for at least 28 consecutive days. In certain embodiments, a composition described herein can be administered to slow down aging and/or support general wellness for subjects over the age of 30, over the age of 40, over the age of 50, over the age of 60, over the age of 70, over the age of 80, or over the age of 90. A composition described herein can be administered in a single dose or multiple doses per day and the total dosage of beta-alanine in the composition for each day of administration may be about 0.01 g to about 200 g, about 0.02 g to about 10 g, about 0.03 g to about 6.4 g, about 0.04 g to about 3.2 g, about 0.05 g to about 2.24 g, about 0.1 g to about 1 g, about 0.2 g to 0.8 g or about 0.3 g to 0.6 g.

The amount of a composition described herein administered can be adjusted daily, weekly, or monthly. In certain embodiments, the amount of a composition administered can be increased daily, every two days, every three days, every four days, every five days, and every six days, or longer. In certain embodiments, the amount of a composition administered can be increased weekly, every two weeks, every three weeks, or every four weeks. In certain embodiments, the amount of a composition administered can be increased daily for about two days, three days, four days, five days, six days, seven days, fourteen days, twenty-one days, twenty-eight days, thirty days, sixty days, or more or any days in between, followed by a constant dosage, a decreased dosage, or a combination thereof. In certain embodiments, a composition can be administered in treatment periods that last for about four weeks. In certain embodiments, a composition that does not contain an anti-inflammatory agent may be administered first followed by administration of an anti-inflammatory agent-containing composition, where the interval between the two administrations can be from about 15 minutes to 24 hours. In certain embodiments, a composition that does not contain an anti-inflammatory agent and an anti-inflammatory agent-containing composition can be administered alternately.

A beta-alanine composition comprising an encapsulated beta-alanine composition and an unencapsulated beta-alanine composition can be administered. In certain embodiments, a composition comprises an unencapsulated beta-alanine and an encapsulated beta-alanine at a total amount of about 0.01 g to about 200 g, about 0.02 g to about 10 g, about 0.03 g to about 6.4 g, about 0.04 g to about 3.2 g, about 0.05 g to about 2.24 g, about 0.1 g to about 1 g, about 0.2 g to 0.8 g or about 0.2 g to 0.4 g, and values therebetween, of beta-alanine per serving. In certain embodiments, a composition comprises an unencapsulated beta-alanine and an encapsulated beta-alanine at a total amount of about 0.1 g, 0.15 g, 0.2 g, 0.25 g, 0.26 g, 0.27 g, 0.28 g, 0.29 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1.0 g, 1.1 g, 1.2 g, 1.6 g, 2.24 g, 2.4 g, 3.2 g, 4.8 g, or 6.4 g, and values therebetween, of an unencapsulated beta-alanine and an encapsulated beta-alanine per serving. The total dosage of an unencapsulated beta-alanine and an encapsulated beta-alanine in a composition for each day may be about 0.01 g to about 200 g, about 0.02 g to about 10 g, about 0.03 g to about 6.4 g, about 0.04 g to about 3.2 g, about 0.05 g to about 2.24 g, about 0.1 g to about 1 g, about 0.2 g to 0.8 g, or about 0.3 g to 0.6 g and values therebetween.

Certain dosing regimens can comprise the administration of alternating dosages of encapsulated and unencapsulated beta-alanine. In certain embodiments, and as one example of alternating days of administration, the total dosage of an unencapsulated beta-alanine and an encapsulated beta-alanine in a composition for an odd number date of a month may be about 0.1 g, 0.15 g, 0.2 g, 0.25 g, 0.26 g, 0.27 g, 0.28 g, 0.29 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1.0 g, 1.1 g, 1.2 g, 1.6 g, 2.24 g, 2.4 g, 3.2 g, 4.8 g, or 6.4 g, and values therebetween, and for an even number date of the month is different from that on the odd date and may be about 0.1 g, 0.15 g, 0.2 g, 0.25 g, 0.26 g, 0.27 g, 0.28 g, 0.29 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1.0 g, 1.1 g, 1.2 g, 1.6 g, 2.24 g, 2.4 g, 3.2 g, 4.8 g, or 6.4 g, and values therebetween. Alternating dosages of encapsulated and unencapsulated beta-alanine can comprise alternating dosages within a single day, every other day, or other variations of alternating dosage regimes that would be immediately envisaged by the skilled artisan in view of the disclosure contained herein. The dosing regimens described herein are not particularly limited and can comprise dosing regimens wherein an encapsulated beta-alanine is taken in the same day as an unencapsulated beta-alanine and regimens wherein unencapsulated beta-alanine is not taken in the same day as an encapsulated beta-alanine, and vice versa.

In certain embodiments, an encapsulated beta-alanine composition and an unencapsulated beta-alanine composition can be administered separately. In certain embodiments, an encapsulated beta-alanine may be administered to a subject at the same time as an unencapsulated beta-alanine. In those instances, the encapsulated beta-alanine and unencapsulated beta-alanine may be administered in a single composition or in more than one composition that are administer in temporal proximity to one another. The relative amounts of encapsulated beta-alanine and unencapsulated beta-alanine can be administered to subjects in any of the amounts and ratios as described herein and those that would be immediately envisaged by the skilled artisan in view of the disclosure contained herein. In certain embodiments, an encapsulated beta-alanine composition may be administered first followed by administration of an unencapsulated beta-alanine composition, where the interval between the two administrations can be from about 15 minutes to 24 hours. In certain embodiments, an unencapsulated beta-alanine composition may be administered first followed by administration of an encapsulated beta-alanine composition, where the interval between the two administrations can be from about 5 minutes to about 24 hours, and any interval therebetween. In certain embodiments, an encapsulated beta-alanine composition and an unencapsulated beta-alanine composition can be administered alternately. In certain embodiments, an encapsulated beta-alanine composition may be administered to a subject at a different time or frequency than an unencapsulated beta-alanine. For example, and without limitation, an encapsulated beta-alanine may be administered to a subject several times per week whereas an unencapsulated beta-alanine may be administered daily. As another example, and without limitation, an unencapsulated beta-alanine may be administered to a subject several times per week whereas an encapsulated beta-alanine may be administered daily. In such instances, the unencapsulated beta-alanine may be present in a sustained release, or other controlled or delayed release formulation. Each form of beta-alanine can be administered at the frequencies described herein. In certain embodiments, an unencapsulated beta-alanine composition and an encapsulated beta-alanine composition independently contain about 0.01 g to about 200 g, about 0.02 g to about 10 g, about 0.03 g to about 6.4 g, about 0.04 g to about 3.2 g, about 0.05 g to about 2.24 g, about 0.1 g to about 1 g, about 0.2 g to 0.8 g, or about 0.2 g to 0.4 g and values therebetween, of beta-alanine. In certain embodiments, an unencapsulated beta-alanine composition and an encapsulated beta-alanine composition independently contain about 0.05 g, 0.1 g, 0.15 g, 0.2 g, 0.25 g, 0.26 g, 0.27 g, 0.28 g, 0.29 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1.0 g, 1.1 g, 1.2 g, 1.6 g, 2.24 g, 2.4 g, 3.2 g, 4.8 g, or 6.4 g, and values therebetween, of beta-alanine. The total dosage of an unencapsulated beta-alanine and an encapsulated beta-alanine from two compositions for each day may be about 0.01 g to about 200 g, about 0.02 g to about 10 g, about 0.03 g to about 6.4 g, about 0.04 g to about 3.2 g, about 0.05 g to about 2.24 g, about 0.1 g to about 1 g, about 0.2 g to 0.8 g, or about 0.3 g to 0.6 g and values therebetween.

Compositions described herein may be administered as the sole treatment or in conjunction with other supplements, vitamins, or drugs useful in enhancing athletic performance and/or reducing paresthesia. Compositions described herein can be administered as the sole treatment or in conjunction with other supplements, vitamins, or drugs to maintain healthy levels of brain activity, cognition, memory, mental acuity, focus, and spatial awareness.

A composition described herein can be administered in a single dose or multiple doses per day. For example, a composition can be administered one time, twice, three times, four times, five times, six times, seven times, eight times or more in a 24-hour period. In certain embodiments, the total dosage of beta-alanine in a composition for a 24-hour period may be about 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1.0 g, 1.1 g, 1.2 g, 1.3 g, 1.4 g, 1.5 g, 2.0 g, 2.1 g, 2.2 g, 2.3 g, 2.4 g, 2.5 g, 2.6 g, 2.7 g, 2.8 g, 2.9 g, 3.0 g, 3.1 g, 3.2 g, 3.3 g, 3.4 g, 3.5 g, 3.6 g, 3.7 g, 3.8 g, 3.9 g, 4.0 g, 4.1 g, 4.2 g, 4.3 g, 4.4 g, 4.5 g, 4.6 g, 4.7 g, 4.8 g, 4.9 g, 5.0 g, 5.1 g, 5.2 g, 5.3 g, 5.4 g, 5.5 g, 5.6 g, 5.7 g, 5.8 g, 5.9 g, 6.0 g, 6.1 g, 6.2 g, 6.3 g, 6.4 g, 6.5 g, 6.75 g, 7.0 g, 7.25 g, 7.5 g, 7.75 g, 8.0 g, 8.25 g, 8.5 g, 8.75 g, 9.0 g, 8.25 g, 9.5 g, 9.75 g, 10 g, 20 g, 30 g, 40 g, 50 g, 60 g, 70 g, 80 g, 90 g, 100 g, 110 g, 120 g, 130 g, 140 g, 150 g, 160 g, 170 g, 180 g, 190 g, 200 g, 500 g, 1000 g, 1500 g, 1600 g, 2000 g, 2500 g, 3000 g, 3200 g, 4000 g, 5000 g, 6000 g, 6400 g or more or any amount therebetween.

The total dosage of an anti-inflammatory agent in a composition for a 24-hour period may be about 0 μg, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 125 μg, 150 μg, 175 μg, 200 μg, 225 μg, 250 μg, 275 μg, 300 μg, 325 μg, 350 μg, 375 μg, 400 μg, 425 μg, 450 μg, 475 μg, 500 μg, 525 μg, 575 μg, 600 μg, 625 μg, 650 μg, 675 μg, 700 μg, 725 μg, 750 μg, 775 μg, 800 μg, 825 μg, 850 μg, 875 μg, 900 μg, 925 μg, 950 μg, 975 μg, 1000 μg, 1 mg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1.25 g, 1.5 g, 1.8 g, 2.0 g, 2.25 g, 2.4 g, 2.5 g, 2.75 g, 3.0 g, 3.25 g, 3.5 g, 3.5 g, 3.75 g, 4.0 g, 4.25 g, 4.5 g, 4.75 g, 5.0 g, 5.25 g, 5.5 g, 5.75 g, 6.0 g, 6.25 g, 6.5 g, 6.75 g, 7.0 g, 7.25 g, 7.5 g, 7.75 g, 8.0 g, 8.25 g, 8.5 g, 8.75 g, 9.0 g, 8.25 g, 9.5 g, 9.75 g, 10 g, or more, or any amount therebetween.

The total dosage of an amphiphilic agent in a composition for a 24-hour period may be about 0 μg, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 125 μg, 150 μg, 175 μg, 200 μg, 225 μg, 250 μg, 275 μg, 300 μg, 325 μg, 350 μg, 375 μg, 400 μg, 425 μg, 450 μg, 475 μg, 500 μg, 525 μg, 575 μg, 600 μg, 625 μg, 650 μg, 675 μg, 700 μg, 725 μg, 750 μg, 775 μg, 800 μg, 825 μg, 850 μg, 875 μg, 900 μg, 925 μg, 950 μg, 975 μg, 1000 μg, 1 mg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1.25 g, 1.5 g, 1.75 g, 2.0 g, 2.25 g, 2.5 g, 2.75 g, 3.0 g, 3.25 g, 3.5 g, 3.5 g, 3.75 g, 4.0 g, 4.25 g, 4.5 g, 4.75 g, 5.0 g, 5.25 g, 5.5 g, 5.75 g, 6.0 g, 6.25 g, 6.5 g, 6.75 g, 7.0 g, 7.25 g, 7.5 g, 7.75 g, 8.0 g, 8.25 g, 8.5 g, 8.75 g, 9.0 g, 8.25 g, 9.5 g, 9.75 g, 10 g, or more, or any amount therebetween.

The total dosage of a stimulant in a composition for a 24-hour period may be about 0 μg, 1 μg, 2 μg, 3 μg, 4 μg, 5 μg, 6 μg, 7 μg, 8 μg, 9 μg, 10 μg, 15 μg, 20 μg, 25 μg, 30 μg, 35 μg, 40 μg, μg, 50 μg, 55 μg, 60 μg, 65 μg, 70 μg, 75 μg, 80 μg, 85 μg, 90 μg, 95 μg, 100 μg, 125 μg, 150 μg, 175 μg, 200 μg, 225 μg, 250 μg, 275 μg, 300 μg, 325 μg, 350 μg, 375 μg, 400 μg, 425 μg, 450 μg, 475 μg, 500 μg, 525 μg, 575 μg, 600 μg, 625 μg, 650 μg, 675 μg, 700 μg, 725 μg, 750 μg, 775 μg, 800 μg, 825 μg, 850 μg, 875 μg, 900 μg, 925 μg, 950 μg, 975 μg, 1000 μg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1.25 g, 1.5 g, 1.75 g, 2.0 g, 2.25 g, 2.5 g, 2.75 g, 3.0 g, 3.25 g, 3.5 g, 3.5 g, 3.75 g, 4.0 g, 4.25 g, 4.5 g, 4.75 g, 5.0 g, 5.25 g, 5.5 g, 5.75 g, 6.0 g, 6.25 g, 6.5 g, 6.75 g, 7.0 g, 7.25 g, 7.5 g, 7.75 g, 8.0 g, 8.25 g, 8.5 g, 8.75 g, 9.0 g, 8.25 g, 9.5 g, 9.75 g, 10 g, or more, or any amount therebetween.

A composition described herein can be given on a daily basis over a period of about 1 day to about four or more weeks. In certain embodiments, the total dosage of beta-alanine in a composition for each day may be about 0.01 g to about 10 g, about 0.02 g to about 6.4 g, about 0.04 g to about 3.2 g, about 0.08 g to about 2.4 g, about 0.1 g to about 2 g, about 0.2 g to about 1.8 g, about 0.3 g to about 0.6 g, about 1.0 g to about 1.8 g, about 0.1 g, about 0.2 g, about 0.54 g, about 0.84 g, about 1.12 g, about 1.2 g, about 1.6 g, about 1.68 g, about 2.34 g, about 2.4 g, about 3.2 g, about 3.36 g, about 4.68 g, about 4.8 g, or about 6.4 g, and values therebetween. In certain embodiments, a composition contains about 0.1 g, 0.15 g, 0.2 g, 0.25 g, 0.27 g, 0.3 g, 0.35 g, 0.4 g, 0.45 g, 0.5 g, 0.55 g, 0.6 g, 0.65 g, 0.7 g, 0.75 g, 0.8 g, 0.84 g, 1.12 g, 1.2 g, 1.6 g, 1.68 g, 2.34 g, 2.4 g, 3.2 g, 3.36 g, 4.68 g, 4.8 g, or 6.4 g, and values therebetween, of beta-alanine. In certain embodiments, the total dosage of an anti-inflammatory agent in a composition for each day may be about 0.001 g to about 1 g, about 0.002 g to about 0.5 g, about 0.002 g to about 0.004 g, about 0.004 g to about 0.008 mg, or about 0.002 g to about 0.25 g, and values therebetween. In certain embodiments, the total dosage of an amphiphilic agent in the composition for each day may be about 0.001 g to about 1 g, about 0.002 g to about 0.5 g, about 0.005 g to about 0.25 g, about 0.012 g to about 0.024 g, or about 0.008 g to about 0.1 g, and values therebetween. The amount of a composition administered can be adjusted daily, weekly, or monthly. In certain embodiments, the amount of a composition administered can be increased daily, every two days, every three days, every four days, every five days, and every six days. In certain embodiments, the amount of a composition administered can be increased weekly, every two weeks, every three weeks, every four weeks, every five weeks, and every six weeks. In certain embodiments, the amount of a composition administered can be increased daily for about three days, four days, five days, six days, seven days, fourteen days, twenty-one days, twenty-eight days, thirty days, sixty days, or more or any days in between followed by a constant dosage, a decreased dosage, or a combination thereof. In certain embodiments, a composition that does not contain an anti-inflammatory agent may be administered first followed by administration of an anti-inflammatory agent-containing composition, where the interval between the two administrations can be from about 15 minutes to 24 hours. In certain embodiments, a composition that does not contain an anti-inflammatory agent and an anti-inflammatory agent-containing composition can be administered alternately. In certain embodiments, the amount of a composition administered can be decreased daily, every two days, every three days, every four days, every five days, and every six days. In certain embodiments, the amount of a composition administered can be decreased weekly.

Compositions described herein may be administered as the sole treatment or in conjunction with other supplements or drugs useful in improving cognitive function.

In some embodiments, an unencapsulated beta-alanine composition may be administered to a subject for a therapeutic or nutraceutical outcome (as described herein) that is different than the therapeutic or nutraceutical outcome that an encapsulated beta-alanine composition is administered to a subject for. In certain embodiments, an encapsulated beta-alanine composition may be administered to a subject for the same purpose as an unencapsulated beta-alanine composition. Each form of beta-alanine can be administered at the frequencies described herein. Compositions described herein may be administered as the sole treatment or in conjunction with other supplements, vitamins, or drugs useful for any of the outcomes described herein.

Certain embodiments demonstrate unexpectedly improved stability, without being bound by any particular theory, due to improved resistance to moisture absorption. Certain embodiments comprise a composition possessing unexpectedly improved stability (e.g., shelf life) wherein the composition remains stable for about 1 month, 2 months, 3 months, 6 months, 12 months, 18 months, or more and values therebetween. Certain embodiments as described herein possess unexpectedly improved particle size distribution and uniformity compared to prior art beta-alanine compositions. For example, certain embodiments can comprise a beta-alanine composition having a particle size distribution wherein greater than about 50%, 75%, 85%, 90%, or 95% and values therebetween of particles pass 425 μm mesh. Certain embodiments as described herein possess unexpectedly improved membrane permeability, and without being bound by any particular theory, the improved membrane permeability allows for improved passage through cellular membranes (e.g., small intestine, muscle cells, and the like) thereby providing unexpectedly improved bioavailability. Certain embodiments comprise a beta-alanine composition wherein the composition has unexpectedly improved bioavailability compared to prior art beta-alanine compositions wherein the composition can be administered orally and achieve the improved bioavailability.

Certain embodiments comprise a beta-alanine composition wherein the beta-alanine composition provides unexpectedly superior suppression of lactate and lactic acid buildup in the muscle, for example, during anaerobic and/or aerobic exercise. Across a range of subjects, certain embodiments, as described herein, comprise a beta-alanine composition wherein the composition provides a more uniform reduction in lactate concentration in subjects compared to prior art beta-alanine compositions. Certain embodiments comprise a beta-alanine composition as described herein wherein the beta-alanine composition is administered to effectively reduce cortisol concentration and/or rate of production in a subject. Certain embodiments comprise a beta-alanine composition as described herein wherein the beta-alanine composition is administered to effectively reduce C-reactive protein concentration and/or rate of production in a subject. Certain embodiments comprise a beta-alanine composition, as described herein, wherein the beta-alanine composition is administered to a subject to increase carnosine production and/or concentration in a subject's tissues, wherein the tissues are selected from the group consisting of skeletal muscle, brain tissue, heart muscle, cells, and combinations thereof.

Certain embodiments comprise a beta-alanine composition, as described herein, wherein the composition is administered to improve and/or maintain a healthy mood in a subject. For example and without limitation, indicators of a healthy and/or improved mood include one or more of tenseness, anger, fatigue (e.g. “worn out”), happiness, pride, liveliness, confusion, sadness, activeness, feelings of being “on-edge,” grouchiness, shame, energy, hopelessness, uneasiness, restlessness, concentration and/or focus, fatigue, competency, annoyance, discouragement, resentfulness, nervousness, feelings of being miserable, confidence, bitterness, exhaustion, anxiety, helplessness, weariness, satisfaction, bewilderment, fury, enthusiasm, worthlessness, forgetfulness, vigor, uncertainty, embarrassment, and the like.

Certain embodiments comprise a beta-alanine composition, as described herein, wherein the beta-alanine composition provides unexpectedly superior improvements in inflammatory markers, e.g. cytokines, interleukins (e.g., IL-6 and IL-10 and the like), and the like, compared to prior art beta-alanine compositions.

Certain embodiments comprise a beta-alanine composition, as described herein, wherein the composition provides unexpectedly superior improves in physical endurance and athletic performance. Without being bound by any particular theory, compositions as described herein can provide unexpectedly improved gains in short-term muscle exertion (e.g., weight lifting, sprinting, muscle twitch speed, and the like) and/or unexpectedly improved gains in long-term muscle exertion (e.g., long-distance running, aerobic activity, swimming, long-distance cycling, and the like).

A method of preparing a composition according to the disclosure contained herein, is exemplified by the following steps:
step (a) adding an amphiphilic agent in water to form a first solution;
step (b) homogenizing the first solution to form a first emulsion;
step (c) adding an anti-inflammatory agent into the first emulsion to form a second solution;
step (d) homogenizing the second solution to form a second emulsion;
step (e) adding beta-alanine into the second emulsion to form a third solution;
step (f) homogenizing the third solution to form a third emulsion;
step (g) adding a stimulant into the third emulsion to form a fourth solution;
step (h) homogenizing the fourth solution to form a fourth emulsion;
step (i) preparing an aqueous solution of a coating agent and adding the aqueous solution into the third emulsion to form a fifth solution;
step (j) homogenizing the fifth solution to form a fifth emulsion; and
step (k) spray-drying the fifth emulsion to form a dried beta-alanine composition.
A beta-alanine composition can also be prepared by the following steps:
step (a) adding an amphiphilic agent in water to form a first solution;
step (b) homogenizing the first solution to form a first emulsion;
step (c) adding an anti-inflammatory agent into the first emulsion to form a second solution;
step (d) homogenizing the second solution to form a second emulsion;
step (e) adding a mixture of beta-alanine and a stimulant into the second emulsion to form a third solution;
step (f) homogenizing the third solution to form a third emulsion;
step (g) adding a water solution of a coating agent into the third emulsion to form a fourth solution;
step (h) homogenizing the fourth solution to form a fourth emulsion; and
step (i) spray-drying the fourth emulsion to form a dried beta-alanine composition.

In certain embodiments, when a composition does not contain an ingredient such as a stimulant, an amphiphilic agent, or an anti-inflammatory agent, the steps involving such an ingredient can be omitted. The disclosure contained herein contemplates that the foregoing method steps can be rearranged as would be envisaged by the skilled artisan in view of the disclosure and claims set forth herein.

The terminology used herein is for the purpose of describing particular embodiments only and is not intended to be limiting. As used in the Specification and the appended claims, the singular forms “a”, “an” and “the” include plural referents unless the context clearly dictates otherwise.

The terms “reducing” is used herein to generally refer to relieving the disease, causing regression of the disease and/or its symptoms, conditions, and co-morbidities and obtaining a desired pharmacological and physiological effect and can also refer to a nutritional or nutraceutical effect, the scopes, and meanings of which will be clear to the skilled artisan based upon the context in which these terms are used. As set forth herein, any composition that is administered to reduce any condition, can also be administered to maintain a healthy level of a physiological or biological condition. In certain embodiments, a composition is administered to maintain a healthy level of one or more of the conditions disclosed herein. The scope and meaning of “reducing” and “maintaining healthy levels of” would be immediately envisaged by the skilled artisan when viewing the term in the context of the disclosure and the claims.

The term “liposomal” and “liposome” described herein can refer to an artificial vesicle having at least one lipid bilayer. The vesicle can be multilamellar large, oligolamellar, small unilamellar, medium-sized unilamellar, large unilamellar, giant unilamellar, and multivesicular.

The term “pharmaceutical composition”, “composition” and the like can refer to preparations which are in such a form as to permit the biological activity of the active ingredients to be effective, and therefore may be administered to a subject for therapeutic use along with dietary and/or nutritional supplement use. The term “emulsion” can refer to a mixture of two or more liquids that are normally immiscible owing to liquid-liquid separation. The term “homogenize” can refer to any processes used to make a mixture of two mutually non-soluble liquids the same throughout by turning one of the liquids into a state consisting of extremely small particles distributed uniformly throughout the other liquid. The term “spray-drying” can refer to a process involving the atomization of a solution, slurry, or emulsion containing one or more components of the desired product into droplets by spraying followed by the rapid evaporation of the sprayed droplets into solid powder by hot air at a certain temperature and pressure. The meaning of these terms will be clear to the skilled artisan based upon the context in which they are used.

As provided herein, the disclosure of a “ratio” of compounds and compositions corresponds to a ratio provided in terms of weight mass of the components present in the ratio.

As used herein, the term “subject” refers to animals, including mammals and humans.

As used herein, attenuating aging process and anti-aging effects are interchangeable.

To provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

### EXAMPLES

### Example 1: Preparation of Beta-Alanine Compositions

### Composition 1

Lecithin (37 g) was dissolved in water (1.16 L) at 70° C. and homogenized for 10 minutes to form an emulsion. To the emulsion, beta-caryophyllene (40 g) was added. The resultant solution was homogenized for 5 minutes, then beta-alanine (820 g) was added to form a solution, which was homogenized for 10 minutes and then caffeine (2 g) was added. The resultant solution was homogenized for 5 minutes, followed by adding a water solution of modified food starch (101 g). The resultant mixture was spray-dried with outlet temperature of 90° C. and inlet temperature of 180° C. to give a dry powder.

The powder (854 g) was blended homogeneously with a mixture of flavor agents (116 g) and sucrose powder (30 g) for 30 minutes to give Composition 1.

### Composition 2

Lecithin (37 g) was dissolved in water (1.16 L) at 70° C. and homogenized for 10 minutes to form an emulsion. To the emulsion, beta-caryophyllene (8 g) in clove oil (40 g) was added. The resultant solution was homogenized for 5 minutes, then beta-alanine (820 g) was added to form a solution. The resultant solution was homogenized for 10 minutes and then caffeine (2 g) was added. The resultant solution was homogenized for 5 minutes, followed by adding a water solution of modified food starch (101 g). The resultant mixture was spray dried with outlet temperature of 90° C. and inlet temperature of 180° C. to give a dry powder. The powder (854 g) was blended homogeneously with a mixture of flavor agents (116 g) and sucrose powder (30 g) for 30 minutes to give Composition 2.

### Example 2: Characteristics of Composition 2

Composition 2 was analyzed in terms of particle sizes, zeta potential, morphology, solubility, stability, and dissolution profiles.

### Particle Sizes Measured by ASTM Mesh:

40 ASTM mesh (i.e., 425 m) was used to test particle size of Composition 2. The test was repeated 7 times and the passing-through composition was collected randomly. It was observed that 95% wt./wt. or more of Composition 2 pass through the mesh, indicating that the particle sizes of Composition 2 are predominantly less than 425 m.

### Particle Sizes and Zeta Potential Measured by Dynamic Light Scattering (DLS) Method

Particle Sizes of Composition 2 were further measured by the DLS method using Zetasizer Nano ZS (from Malvern Panalytical). The DLS method and Zetasizer Nano ZS were also used to determine zeta potential of Composition 2. The Composition was appropriately diluted with water and then evaluated at 25° C. to determine its particle sizes and zeta potential. The Stokes-Einstein equation below was used to calculate particle sizes. Zeta potential was determined using the Nano DTS software (version 6.34). Each measurement was carried out in a minimum of three sets of 10 runs.

D=μ_{q}k_{B}T/q (the Stokes-Einstein equation)

where D is the diffusion coefficient, μ_{q} is the electrical mobility of the charged particle, k_{B} is the Boltzmann constant, and T is the absolute temperature. The results are shown in FIGS. 1A and 1B.

FIG. 1A shows that Composition 2 had particle sizes ranging between 109.4 nm and 237.5 nm with an average particle size of 171.3±4.6 nm. FIG. 1A also shows that Composition 2 particles were in homogeneous size distribution and thus were uniform.

FIG. 1B shows that Composition 2 exhibited a negative surface charge of −31.2 mV. It is known in the art that a zeta-potential of greater than ±30 mV is beneficial to a composition's physical stability as aggregation among the composition's particles is prevented due to electrostatic repulsion. This high negative zeta potential of Composition 2 demonstrates the strong electrostatic repulsion among the particles, which prevents their aggregation, indicating their stability. This high negative zeta potential of Composition 1 also supports the homogeneous size distribution of the particles.

The results shown in FIGS. 1A and 1B indicate that compositions described herein are soluble in water. As such, compositions described herein can be formulated as ready-to-make products (e.g., a powder, a capsule, and a tablet) that can be mixed with water before consumption.

### Morphology by Scanning Electron Microscope (SEM)

A SEM (Vega3Tescan, Czech Republic) was used to determine the morphology of Composition 2. Composition 2 was scanned at a 5 kV accelerating voltage after being sputter-coated with a small layer of gold by a sputter gold coater and kept in double-sided carbon ribbon-wrapped aluminium stubs. Histogram images were taken from three different magnifications (i.e., 500 nm, 200 nm, and 50 nm) and particle size distribution was also determined. The results in FIGS. 1C (i)-(iii) show that Composition 2 particles had smooth surfaces and were in spherical forms, free of aggregation. FIG. 1C (iv) shows that the particles were uniformly distributed with an average particle size of about 150 nm. The results, consistent with the results shown in FIGS. 1A and 1B, further indicate that compositions described herein possess unexpectedly improved stability and thus, they can be formulated as ready-to-make products (e.g., a powder, a capsule, and a tablet) that can be mixed with water before consumption.

### Solubility:

To Composition 2 (0.5 mg) was added 100 mL water. It was observed that the composition was dissolved in water instantly and a clear solution was formed, demonstrating that the composition is soluble in water, which is consistent with the results shown in FIGS. 1A, 1B, and 1C (i)-(iv).

### Stability:

The stability of Composition 2 was evaluated as compared to a reference product (from Natural Alternatives International Inc.) containing free unencapsulated beta-alanine (“Reference Product”).

Both Composition 2 and the Reference Product were stored inside two low density polyethylene covers at 40° C. and under relativity humidity of 65%. Every 30 days, Composition 2 and the Reference Product were analyzed for visual appearance, beta-alanine content, moisture content, and bacterial content. The results are shown in Tables 1 and 2 below.

TABLE 1
Composition 2

| | Visual | Beta-alanine | Moisture | Bacterial |
|---|---|---|---|---|
| Day | Appearance | Content (%) | Content (%) | Content |
| 0 | Off-white Color | 70.56 | 2.42 | Not Detected |
| 30 | Off-white Color | 70.64 | 2.61 | Not Detected |
| 60 | Off-white Color | 70.51 | 2.85 | Not Detected |

TABLE 2
Reference Product

| | Visual | Beta-alanine | Moisture | Bacterial |
|---|---|---|---|---|
| Day | Appearance | Content (%) | Content (%) | Content |
| 0 | White Color | 99.4 | 0.5 | Not Detected |
| 30 | White Color | 98.82 | 0.8 | Not Detected |
| 60 | White Color | 98.46 | 1.23 | Not Detected |

The results show that, while the visual appearance and bacterial contents of Composition 2 and the Reference Product did not change after 60 days, the moisture content of Composition 2 increased only 18%, while the moisture content of the Reference Product increased by 146%. The results demonstrate that Composition 2 was unexpectedly more stable than the Reference Product.

The stability of Composition 2 was further evaluated under different storage conditions in a humidity chamber, i.e., 4±2° C. (refrigeration), 25±2° C., and 40±2° C. under 75±5% relativity humidity, all in compliance with International Council for Harmonisation of Technical Requirements for Pharmaceuticals for Human Use (ICH) guidelines. Glass containers were used to hold the samples during the course of the study. The beta-alanine content was measured before storage and every 30 days thereafter. All measures were executed in triplicate. The stability was calculated using the following formula:

$$Stability of β - alanine ( \% ) = \frac{{BA}_{t}}{{BA}_{0}} \times 100$$

The results are shown in Table 3 below.

TABLE 3

| Storage | Stability of beta-alanine (%) | | | | | | |
|---|---|---|---|---|---|---|---|
| Condition | Initial | 30 days | 60 days | 90 days | 120 days | 150 days | 180 days |
| 4 ± 2° C. | 99.62 ± | 99.18 ± | 99.01 ± | 98.94 ± | 98.88 ± | 98.79 ± | 98.71 ± |
| | 0.27 | 0.22 | 0.21 | 0.18 | 0.26 | 0.24 | 0.19 |
| 25 ± 2° C. | 99.62 ± | 99.07 ± | 98.89 ± | 98.72 ± | 98.65 ± | 98.45 ± | 98.36 ± |
| | 0.27 | 0.31 | 0.14 | 0.31 | 0.23 | 0.17 | 0.24 |
| 40 ± 2° C. | 99.62 ± | 98.82 ± | 98.46 ± | 98.32 ± | 98.14 ± | 98.09 ± | 98.04 ± |
| | 0.27 | 0.36 | 0.32 | 0.31 | 0.42 | 0.35 | 0.37 |

The results demonstrate that a composition described herein is stable and can be stored under refrigeration, at room temperature, or at 40° C. for at least six months.

### Example 3: Permeability Study

The bioavailability of Composition 2 was evaluated by an in vitro permeability study using Caco-2 permeability assay as compared to the Reference Product containing free unencapsulated beta-alanine.

### Cell Line and Culture Medium

In 96-well plates (Tarsons India Pvt. Ltd., Kolkata, India), Caco-2 cells (National Centre for Cell Sciences, Pune, India) were cultured with Dulbecco's modified Eagle's medium supplemented with 10% inactivated Fetal Bovine Serum, penicillin (100 IU/ml), streptomycin (100 g/ml), and amphotericin B (5 g/ml) at 37° C. in a humidified environment of 5% CO₂. Trypsin Phosphate Versene Glucose (0.2% trypsin, 0.5% PVP, 0.02% EDTA, and 0.05% glucose in PBS) was used to detach cells from the wells.

### Cytotoxicity Study

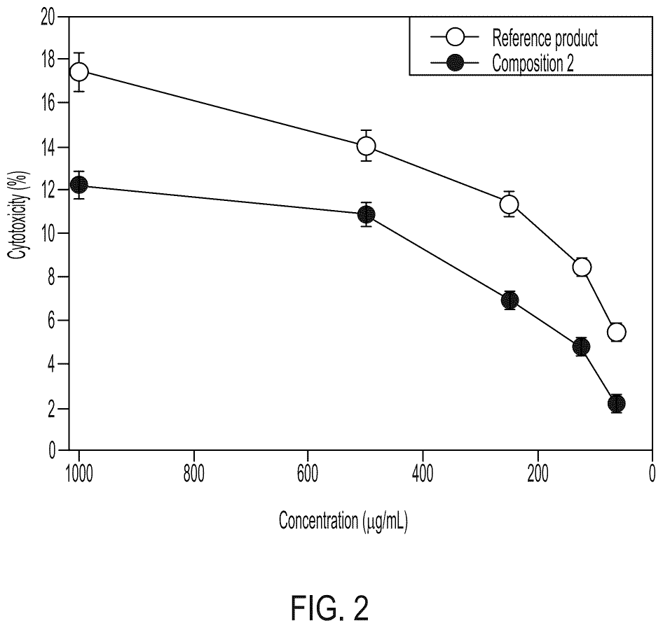

Composition 2 and the Reference Product were evaluated for their in vitro cytotoxicity against Caco-2 cells by using the 3-(4,5-dimethylthiazol-2-yl)-2,5-diphenyl-2H-tetrazolium bromide (MTT) assay in semi-confluent monolayer cultures. This study was conducted to ensure the accuracy of cell permeability results; namely, increased cell permeability was not caused by damages of cells. The cells were treated with a vehicle, Composition 2 or the Reference Product at various concentrations, then incubated at 37° C. under a 5% CO₂ environment for 72 hours. After that, the media was aspirated from the wells, and 100 μl of MTT in Dulbecco's phosphate-buffered saline was added to each well. The cells were incubated for another three hours at 37° C. under a 5% CO₂ environment, during which formazan crystals were formed. The MTT solution was aspirated. Dimethyl sulfoxide (DMSO) was added to solubilize the formazan crystals. The absorbance of the wells was then measured at 540 nm. Dose-response curves and the common toxicity criteria 50 (CTC₅₀; defined as the concentration of a substance killing 50% of total cells/inhibit 50% of the cell growth) values were calculated from the results. The dose-response curves are shown in FIG. 2.

The results show that both Composition 2 and the Reference Product are safe at high doses and Composition 2 had a lower percentage of cytotoxicity than the Reference Product.

### Caco-2 Permeability Study

Caco-2 cells between passages 30 and 35 were seeded on polyester membrane inserts (113.1 mm, 0.4 m pore size) at a density of 100,000 cells per insert and cultured at 37° C. for 25 days, applying culture medium changes three times a week to allow the formation of a confluent monolayer. The integrity of the monolayer was assessed using Lucifer Yellow (LY) dye to determine the apparent permeability coefficient (LY Pₐₚₚ) by the formula below:

$${P}_{app} = \frac{\frac{dQ}{dT}}{( A ) ( Cd )}$$

dQ/dT is the rate of appearance of LY(μg/sec) in the basolateral layer, A is an area of the semi-permeable membrane, and Cd is initial concentration of LY in the apical layer.

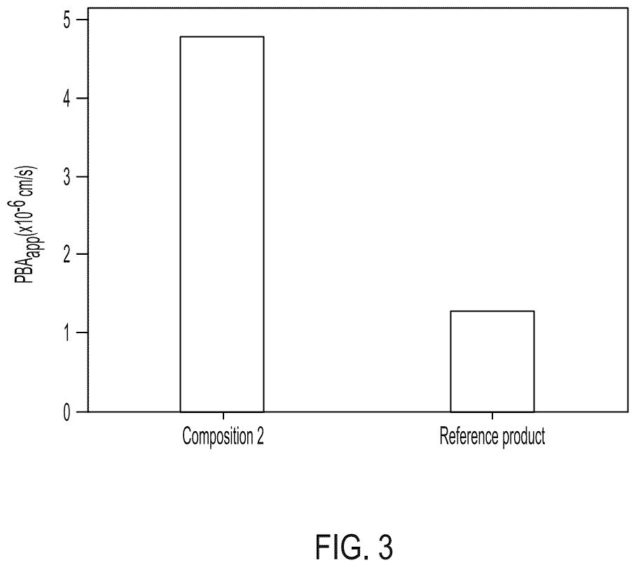

A monolayer having a Pₐₚₚ of less than 0.2×10⁻⁶ cm/sec was used for the permeability study. To mimic in vivo conditions, the pH value of the apical layer was adjusted to 6.0 and the pH value of the basolateral layer was adjusted to 7.4 by using Hank's balanced salt solution (HBSS). A DMSO solution of Composition 2 and a DMSO solution of the Reference Product were diluted with HBSS to give a concentration of 62.5 μg/ml. Each DMSO solution was added to an individual apical layer. The cells were then incubated for 2 hours at 37° C. within an orbital shaker at 100 rpm. At selected time points, 100 μL aliquots were collected from both the apical layer and the basolateral layer and replaced with an equal volume of HBSS. Each aliquot was analyzed by HPLC to quantify the beta-alanine content therein. The following formula was used to calculate the permeability coefficient (PBAₐₚₚ):

$${PBA}_{app} = \frac{Δ Q}{Δ T} \times \frac{1}{A * {C}_{0} * 60}$$

where ΔQ/ΔT is the steady state flux of beta-alanine (mol/sec), A is the surface area of the filter (mm²), and C₀ is the initial concentration of beta-alanine in the Composition 2/Reference Product sample applied in the apical layer (mol/ml). The results are shown in FIG. 3 and Table 4 below.

TABLE 4

| Test Sample | PBAₐₚₚ (×10⁻⁶; cm/s; apical to basolateral) |
|---|---|
| Composition 2 | 4.76 |
| Reference Product | 1.26 |

The results show that Composition 2 unexpectedly exhibited about 3.8 times higher permeability than the reference product. Because higher permeability is associated with better bioavailability, the results indicate, in conjunction with the other aspects described herein, that Composition 2 would be unexpectedly more bioavailable than the Reference Product, i.e., free beta-alanine.

The integrity of the monolayers was assessed at the end of the study to ensure that the differences in permeability were not caused by a loss in monolayer integrity. The monolayers were washed twice with HBSS at pH 7.4 and incubated for 30 minutes at 37° C. HBSS was then added into the apical layer and each solution was added individually to the basolateral layer. It was observed that the integrity of the monolayers treated with Composition 2 or the Reference Product remained unchanged.

### Example 4: Oral Bioavailability Study and Psychological Effects

### Oral Bioavailability Study

The oral bioavailability of a beta-alanine composition described herein was evaluated in a double-blind, randomized, single-dose, three-treatment, three-way crossover study in healthy human adults under fasting conditions as compared to conventional beta-alanine compositions. Twelve healthy human adults (mean±SD age of 52.50±4.52 years) with a body mass index (BMI) ranging between 18.50 kg/m² and 24.99 kg/m² (BMI=weight (kg)/[height (m)]²; mean±SD height of 164.69±8.59 cm and mean±SD weight of 58.11±2.98 Kg) were enrolled in the study and randomly divided into three treatment groups with four participants in each group in a sequential manner. Group 1 was initially dosed orally with Composition 2 (containing 400 mg of encapsulated beta-alanine), Group 2 was initially dosed orally with reference product I (containing 400 mg of conventional beta-alanine, i.e., unencapsulated beta-alanine) and Group 3 was initially dosed orally with reference product II (containing 1200 mg of conventional beta-alanine, i.e., unencapsulated beta-alanine). After an interim washout period of seven days, Group 1 was dosed with reference product I, Group 2 was dosed with reference product II, and Group 3 was dosed with Composition 2. After another interim washout period of seven days, Group 1 was dosed with reference product II, Group 2 was dosed with Composition 2, and Group 3 was dosed with reference product I. At the end of the study, each participant was dosed with Composition 2, reference product I, and reference product II (i.e., each participant had three treatments). Composition 2, reference product I, and reference product II each were formulated into a capsule having a hydroxypropyl methyl cellulose vegetarian shell. All the participants were fasted for at least ten hours before each dosing. In the morning and within one hour before dosing, a 5 mL blood sample from each participant was collected. Each participant was orally administered with its assigned product with 240 mL of water. Post-dose blood samples from each participant were collected at hour 0.25, hour 0.5, hour 1, hour 2, hour 4, hour 6, and hour 8 (5 mL each time) and placed in vacuum tubes containing K₂EDTA. At hour 4 after dosing, each participant was provided with a standardized vegan meal. Beta-alanine concentrations (μmol/L) in plasma were determined by Liquid Chromatography Mass Spectrometry (LC-MS). Each sample (0.1 mL) was mixed with 1.9 mL 0.1% formic acid in water, vortexed for 1 minute, and then centrifuged for 5 minutes. The resultant supernatant was filtered through a 0.22 m syringe filter to give a clear solution, which was directly injected into a LC-MS system (Agilent Technologies 1260 infinity coupled with an Agilent Technologies 6120 Quadrupole mass spectrometer). The HPLC column (Agilent Eclipse plus C-18 column; 150 mm×4.5 mm×3.1 μm) was conditioned by heating to 35° C. using isocratic method with mobile phase A (60% of 0.1% formic acid in water) and mobile phase B (40% 0.1% formic acid in acetonitrile) at a flow rate of 0.8 mL/min with a run time of 10 min. After conditioning, a mobile phase of 0.1% formic acid in acetonitrile and 0.1% formic acid in water was used to run the LC-MS system to give the mass spectra of beta-alanine. Statistical analyses were performed to determine pharmacokinetic (PK) parameters Cₘₐₓ (μmol/L), Kₑₗ, t_{1/2}, (h), AUC₀₋ₜ (μmol·h/L), AUC_{0-∞} (μmol·h/mL), and Tₘₐₓ (h) using SPSS statistical software version 16, expressed as the mean±SD and a p value <0.05 was considered statistically significant. The paired t-test and ANOVA were used to measure the differences among the three-treatment groups, i.e., the same 12 participants treated with Composition 2, reference product I and reference product II described herein.

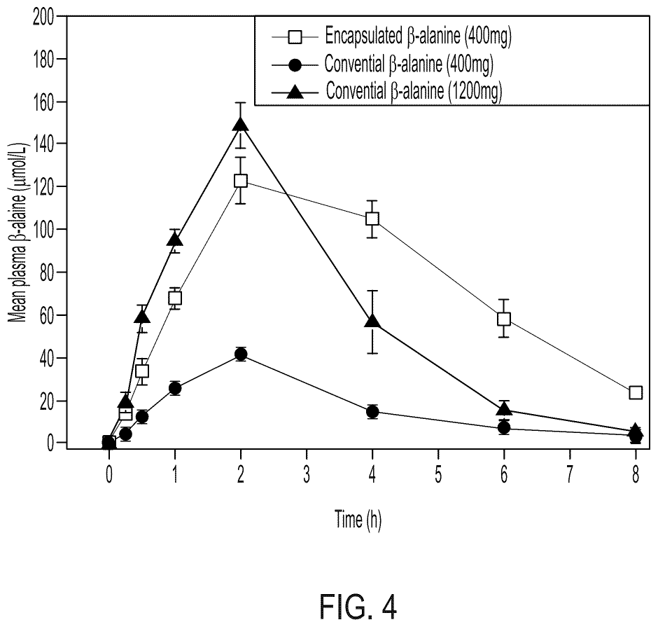

Cₘₐₓ is defined as the maximum plasma concentration. Kₑₗ is defined as an elimination rate constant calculated by the following formula:

$${K}_{el} = ( ln ( {C}_{1} ) - ln ( {C}_{2} ) ) / ( {t}_{2} - {t}_{1} )$$

where C₁ is concentration at time 1 (t₁) and C₂ is concentration at time 2 (t₂). t_{1/2} is defined as the elimination or terminal half-life calculated as 0.693/Kₑₗ. AUC₀₋ₜ is defined as the area under the plasma concentration versus time curve from time zero to last measurable concentration as calculated by linear trapezoidal method. AUC_{0-∞} is defined as the area under the plasma concentration versus time curve from time zero to infinity and was calculated by the following formula:

$${AUC}_{0 - ∞} = {AUC}_{0 - t} + {C}_{t} {K}_{el}$$

where Cₜ is the last measurable concentration and AUC₀₋ₜ and Kₑₗ are defined as above. AUC is an indicator of bioavailability. The higher the AUC, the higher the bioavailability. Tₘₐₓ is defined as the time to reach the maximum plasma concentration. The results are shown in FIG. 4 and Tables 5 and 6 below.

TABLE 5

| | Cₘₐₓ (Mean ± SD) of 12 | Cₘₐₓ (Mean ± SD) of 12 | Cₘₐₓ (Mean ± SD) of 12 |
|---|---|---|---|
| | subjects administered | subjects administered with | subjects administered with |
| | with Composition 2 | reference product I | reference product II |
| | containing 400 mg of | containing 400 mg of | containing 1200 mg of |
| Time | encapsulated beta- | conventional beta-alanine | conventional beta-alanine |
| (h) | alanine (μmol/L) | (μmol/L) | (μmol/L) |
| 0.00 | 0.00 ± 0.00 | 0.00 ± 0.00 | 0.00 ± 0.00 |
| 0.25 | 13.41 ± 2.09 | 4.32 ± 1.11 | 18.97 ± 4.97 |
| 0.50 | 33.58 ± 5.92 | 12.34 ± 2.12 | 58.38 ± 6.05 |
| 1.00 | 67.90 ± 4.90 | 25.74 ± 2.78 | 94.64 ± 5.25 |
| 2.00 | 123.09 ± 10.83 | 41.91 ± 3.11 | 148.79 ± 10.44 |
| 4.00 | 105.04 ± 8.72 | 15.03 ± 2.27 | 56.77 ± 14.75 |
| 6.00 | 58.36 ± 8.76 | 6.72 ± 1.04 | 15.31 ± 4.37 |
| 8.00 | 23.43 ± 2.67 | 3.74 ± 0.30 | 5.29 ± 1.72 |

TABLE 6

| | | 12 subjects | 12 subjects |
|---|---|---|---|
| | 12 subjects | administered with | administered with |
| | administered with | the reference | the reference |
| | Composition 2 | product I | product II |
| | containing 400 mg | containing 400 mg | containing 1200 mg |
| | of encapsulated | of conventional | of conventional |
| PK parameters | beta-alanine | beta-alanine | beta-alanine |
| Cₘₐₓ (μmol/L) | 123.09 ± 10.83* | 41.91 ± 3.11* | 148.79 ± 10.44* |
| AUC₀₋ₜ = AUC_{0-8 h} | 601.75** | 135.11** | 470.25** |
| (μmol · h/L) | | | |
| AUC_{0-∞} | 681.50 | 144.41** | 478.32** |
| (μmol · h/mL) | | | |
| Tₘₐₓ (h) | 2.00 | 2.00 | 2.00 |
| t_{1/2} (h) | 2.36 | 1.72 | 1.06 |
| Kₑₗ | 0.29 | 0.40 | 0.66 |

*p < 0.01 and
**p < 0.001

FIG. 4, Table 5, and Table 6 show that the AUC₀₋₈ₕ and AUC_{0-∞} of beta-alanine in the 12 participants administered with Composition 2 described herein were respectively 601.75 μmol·h/L and 681.50 μmol·h/L, more than 4 times higher than the AUC₀₋₈ₕ and AUC_{0-∞} of beta-alanine in the same 12 participants administered with reference product I (which were respectively 135.11 μmol·h/L and 144.41 μmol·h/L), and significantly higher than the AUC₀₋₈ₕ and AUC_{0-∞} of beta-alanine in the same 12 participants administered with reference product II (which were respectively 470.25 μmol·h/L and 478.32 μmol·h/L). Table 5 shows that administration of 400 mg of encapsulated beta-alanine provided unexpectedly similar increases in Cₘₐₓ values compared to conventional unencapsulated beta-alanine, even when the unencapsulated beta-alanine was administered at a dose three times greater than the encapsulated beta-alanine. Such results are unexpected and demonstrate that a composition as described herein has significantly higher bioavailability than conventional beta-alanine (i.e., unencapsulated) at the same dosage and at a dosage of three times higher.

### Paresthesia and Mood Effects

The three test compositions were evaluated for their paresthesia sensation and mood effects using Visual Analogue Scale (VAS) questionnaires, Qualitative Light Symptoms Inventory (QLSI) questionnaires, and Session-dependent questionnaires in each of the 12 participants. The evaluation was conducted concurrently with each blood sample collection. Pre-dose data shows no paresthesia in each participant. In this double-blind, randomized, single-dose, three-treatment, three-crossover study with two interim washout periods, post-dose scores (mean±SD) were calculated for the 12 participants administered with Composition 2, reference product I, and reference product II between hour 0.25 and hour 8 post-dose and shown in the tables below and figures as identified below.

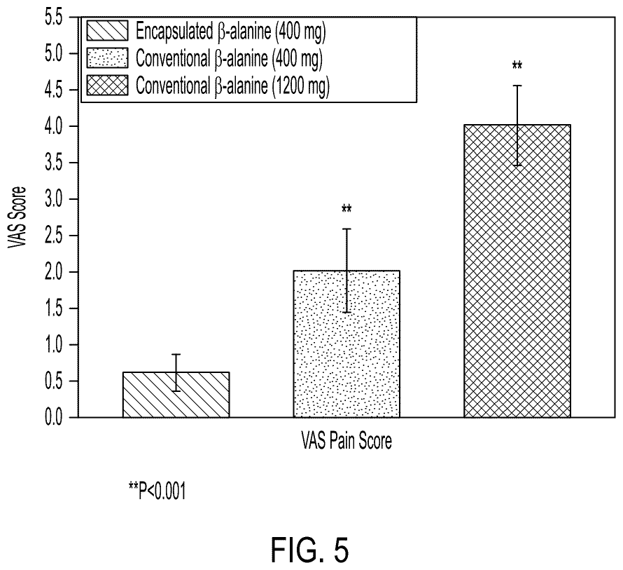

VAS questionnaires assess perceived paresthesia sensation intensity. It consists of a horizontal, continuous 9-cm line with vertical marks 2 mm from each end, labelled from “no unusual sensation” to “most intense sensation imaginable”. A higher score indicates greater paresthesia sensation intensity. A score of 0-2 indicates no sensation to mild sensation and scores 2-4 indicate mild sensation to moderate sensation. Paired t-test was used to compare reference product I and reference product II with Composition 2 at p<0.05 level. The results in Table 7 below and FIG. 5 show that the VAS score in the 12 participants administered with Composition 2 was unexpectedly significantly lower than those in the same 12 participants administered with reference product I and reference product II and that the same 12 participants administered with Composition 2 experienced no paresthesia sensation, the same 12 participants administered with reference product I experienced mild paresthesia sensation, and the same 12 participants administered with reference product II experienced moderate paresthesia sensation. The results demonstrate that a composition described herein does not cause paresthesia as compared to compositions containing the same amount of beta alanine and three times amount of beta alanine.

TABLE 7

| | 12 subjects | | |
|---|---|---|---|
| | administered with | 12 subjects | 12 subjects |
| | Composition 2 | administered with | administered with |
| | described herein | reference product I | reference product II |
| | containing 400 mg | containing 400 mg | containing 1200 mg |
| | of encapsulated | of conventional | of conventional |
| | beta-alanine | beta-alanine | beta-alanine |
| VAS (mean ± SD) | 0.62 ± 0.25 | 2.02 ± 0.57 | 4.01 ± 0.55* |

*p < 0.001 compared to Composition 2

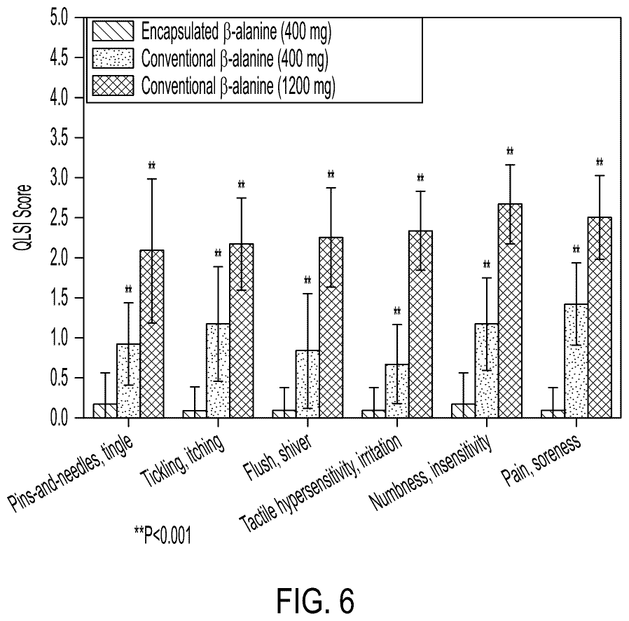

The QLSI questionnaires assess the nature of paresthesia sensation from a subset of six descriptive attributes, i.e., “pins and needles and/or tingles”, “tickling and/or itching”, “flush and/or shiver”, “tactile hypersensitivity and/or irritation”, “numbness and/or insensitivity”, and “pain and/or soreness”. Paresthesia sensation is scaled at 5 levels, with 0 being absent, 1 being mild, 2 being moderate, 3 being strong, and 4 being extremely strong. Participants recorded scores for each sensation. Paired t-test was used to compare reference product I and reference product II with Composition 2 at p<0.05 level. The data are set forth in Table 8 below and FIG. 6. The QLSI results show that the scores in all the sensations recorded by the 12 participants administered with the exemplary composition described herein are significantly lower than those in the same 12 participants administered with reference product I and reference product II and that the 12 participants administered with the exemplary composition described herein experienced no paresthesia sensation and by contrast, the same 12 participants administered with reference product I experienced mild paresthesia sensation and the same 12 participants administered with reference product II experienced moderate to strong paresthesia sensation. The results further demonstrate that a composition described herein does not cause paresthesia as compared to the same amount of free beta-alanine and three times amount of free beta-alanine.

TABLE 8

| | 12 subjects | | |
|---|---|---|---|
| | administered with | 12 subjects | 12 subjects |
| | Composition 2 | administered with | administered with |
| | described herein | reference product I | reference product II |
| | containing 400 mg of | containing 400 mg of | containing 1200 mg of |
| QLSI descriptive | encapsulated beta- | conventional beta- | conventional beta- |
| attributes | alanine (score)^{#} | alanine (score) | alanine (score) |
| pins and needles | 0.17 ± 0.39 | 0.92 ± 0.51** | 2.08 ± 0.90** |
| and/or tingles | | | |
| tickling and/or itching | 0.08 ± 0.29 | 1.17 ± 0.72** | 2.17 ± 0.58** |
| flush and/or shiver | 0.08 ± 0.29 | 0.83 ± 0.72** | 2.25 ± 0.62** |
| tactile hypersensitivity | 0.08 ± 0.29 | 0.67 ± 0.49** | 2.33 ± 0.49** |
| and/or irritation | | | |
| numbness and/or | 0.17 ± 0.39 | 1.17 ± 0.58** | 2.67 ± 0.49** |
| insensitivity | | | |
| pain and/or soreness | 0.08 ± 0.39 | 1.42 ± 0.51** | 2.50 ± 0.52** |

**p < 0.001 compared to Composition 2;
^{#}because most of the participants administered with Composition 2 scaled their sensation as “0”, the standard deviations are greater than the means.

Session-dependent questionnaires, including Profile of Mood States (POMS), Questionnaire de Douleur de Saint-Antoine (QDSA), and the Spielberger State Trait Anxiety Inventory (STAI), were also recorded by each participant. POMS is a French adaptation of the abridged profile of mood, QDSA is a validated French version of the McGill Pain Questionnaire that describes sensory and emotional dimensions of pain, and STAI monitors mood and anxiety. For each questionnaire, paired t-test was used to compare reference product I and reference product II with Composition 2 at p<0.05 level. The higher the score, the more each participant experienced that particular mood state.

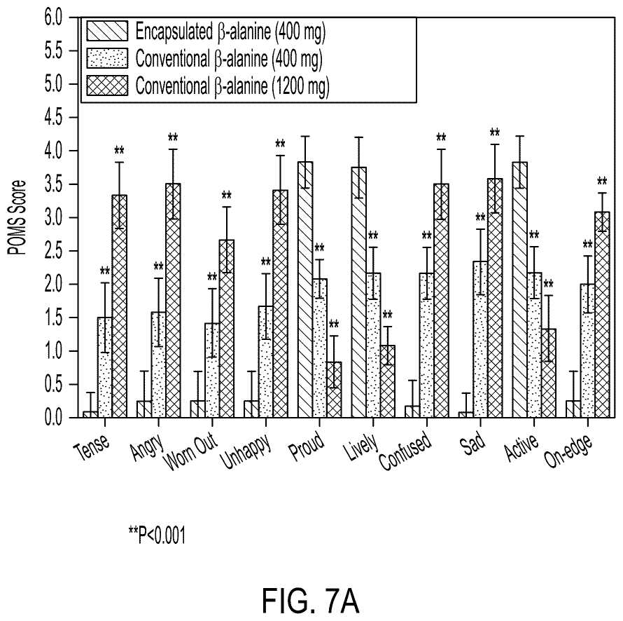

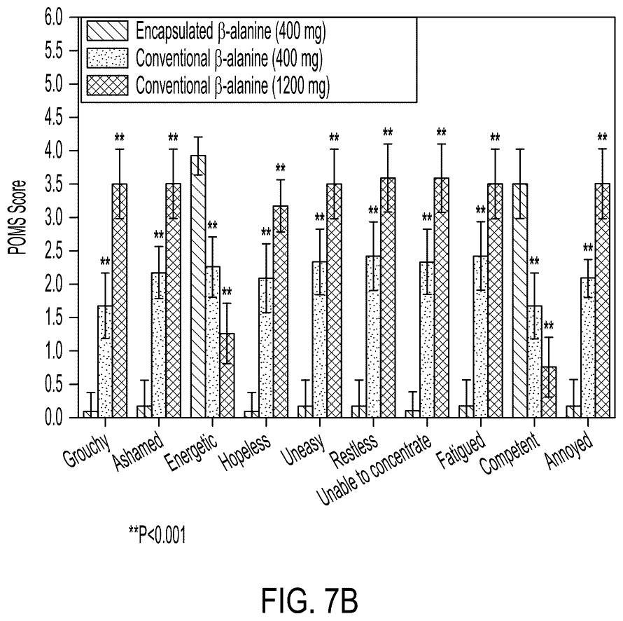

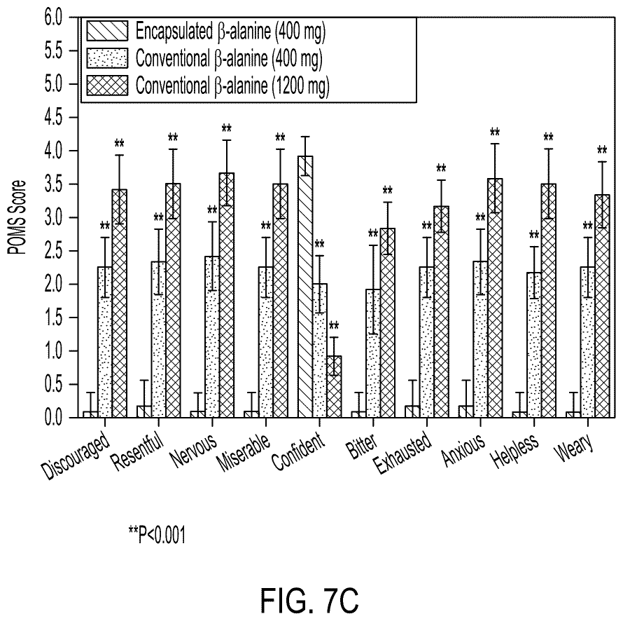

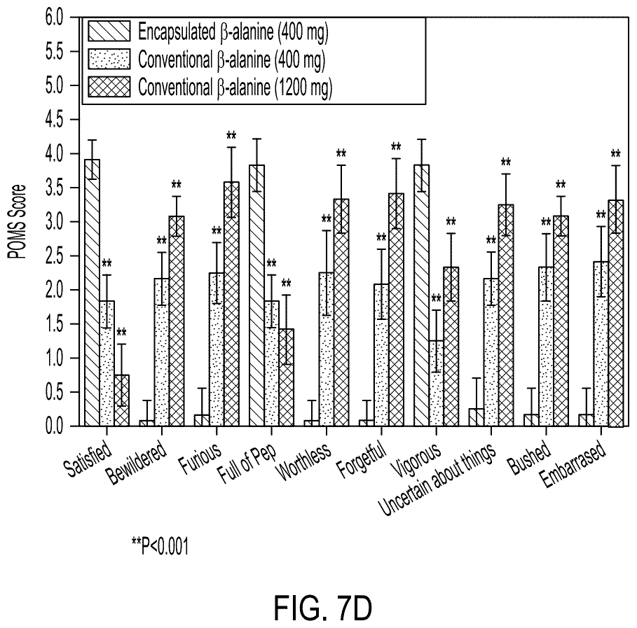

In POMS, participants recorded their mood states at a scale of 0-4, with 0 being not at all, 1 being a little, 2 being moderately, 3 being quite a lot, and 4 being extremely. The POMS results in Table 9 and FIGS. 7A-7D show that the 12 participants administered with Composition 2 described herein scored significantly higher in positive moods (such as proud, active, energetic, competent, confident, satisfied, full of pep, and vigorous) than the same 12 participants administered with reference product I and reference product II, while the 12 participants administered with reference product I and with reference product II scored significantly higher in negative moods (such as tense, angry, worn out, unhappy, confused, sad, no-edge, grouchy ashamed, hopeless, uneasy, restless, unable to concentrate, fatigued, annoyed, discouraged, resentful, nervous, miserable, bitter, exhausted, anxious, helpless, weary, bewildered, furious, worthless, forgetful, uncertain about things, bushed, and embarrassed) than the same 12 participants administered with Composition 2 described herein. The results indicate that a composition as described herein can be used to promote a healthy mood and maintain healthy mood parameters.

TABLE 9

| | 12 subjects | | |
|---|---|---|---|
| | administered with | 12 subjects | 12 subjects |
| | Composition 2 | administered with | administered with |
| | described herein | reference product I | reference product II |
| | containing 400 mg | containing 400 mg | containing 1200 mg |
| | of encapsulated | of conventional | of conventional |
| POMS Parameters | beta-alanine (score)^{#} | beta-alanine (score) | beta-alanine (score) |
| Tense | 0.08 ± 0.29 | 1.50 ± 0.52** | 3.33 ± 0.49** |
| Angry | 0.25 ± 0.45 | 1.58 ± 0.51** | 3.50 ± 0.52** |
| Worn Out | 0.25 ± 0.45 | 1.42 ± 0.51** | 2.67 ± 0.49** |
| Unhappy | 0.25 ± 0.45 | 1.67 ± 0.49** | 3.42 ± 0.51** |
| Proud | 3.83 ± 0.39 | 2.08 ± 0.29** | 0.83 ± 0.39** |
| Lively | 3.75 ± 0.45 | 2.17 ± 0.39** | 1.08 ± 0.29** |
| Confused | 0.17 ± 0.39 | 2.17 ± 0.39** | 3.50 ± 0.52** |
| Sad | 0.08 ± 0.29 | 2.33 ± 0.49** | 3.58 ± 0.51** |
| Active | 3.83 ± 0.39 | 2.17 ± 0.39** | 1.33 ± 0.49** |
| On-edge | 0.25 ± 0.45 | 2.00 ± 0.43** | 3.08 ± 0.29** |
| Grouchy | 0.08 ± 0.29 | 1.67 ± 0.49** | 3.50 ± 0.52** |
| Ashamed | 0.17 ± 0.39 | 2.17 ± 0.39** | 3.50 ± 0.52** |
| Energetic | 3.92 ± 0.29 | 2.25 ± 0.45** | 1.25 ± 0.45** |
| Hopeless | 0.08 ± 0.29 | 2.08 ± 0.51** | 3.17 ± 0.39** |
| Uneasy | 0.17 ± 0.39 | 2.33 ± 0.49** | 3.50 ± 0.52** |
| Restless | 0.17 ± 0.39 | 2.42 ± 0.51** | 3.58 ± 0.51** |
| Unable to concentrate | 0.08 ± 0.29 | 2.33 ± 0.49** | 3.58 ± 0.51** |
| Fatigued | 0.17 ± 0.39 | 2.42 ± 0.51** | 3.50 ± 0.52** |
| Competent | 3.50 ± 0.52 | 1.67 ± 0.49** | 0.75 ± 0.45** |
| Annoyed | 0.17 ± 0.39 | 2.08 ± 0.29** | 3.50 ± 0.52** |
| Discouraged | 0.08 ± 0.29 | 2.25 ± 0.45** | 3.42 ± 0.51** |
| Resentful | 0.17 ± 0.39 | 2.33 ± 0.49** | 3.50 ± 0.52** |
| Nervous | 0.08 ± 0.29 | 2.42 ± 0.51** | 3.67 ± 0.49** |
| Miserable | 0.08 ± 0.29 | 2.25 ± 0.45** | 3.50 ± 0.52** |
| Confident | 3.92 ± 0.29 | 2.00 ± 0.43** | 0.92 ± 0.29** |
| Bitter | 0.08 ± 0.29 | 1.92 ± 0.67** | 2.83 ± 0.39** |
| Exhausted | 0.17 ± 0.39 | 2.25 ± 0.45** | 3.17 ± 0.39** |
| Anxious | 0.17 ± 0.39 | 2.33 ± 0.49** | 3.58 ± 0.51** |
| Helpless | 0.08 ± 0.29 | 2.17 ± 0.39** | 3.50 ± 0.52** |
| Weary | 0.08 ± 0.29 | 2.25 ± 0.45** | 3.33 ± 0.49** |
| Satisfied | 3.92 ± 0.29 | 1.83 ± 0.39** | 0.75 ± 0.45** |
| Bewildered | 0.08 ± 0.29 | 2.17 ± 0.39** | 3.08 ± 0.29** |
| Furious | 0.17 ± 0.39 | 2.25 ± 0.45** | 3.58 ± 0.51** |
| Full of energy/or | 3.83 ± 0.39 | 1.83 ± 0.39** | 1.42 ± 0.51** |
| enthusiasm | | | |
| Worthless | 0.08 ± 0.29 | 2.25 ± 0.62** | 3.33 ± 0.49** |
| Forgetful | 0.08 ± 0.29 | 2.08 ± 0.51** | 3.42 ± 0.51** |
| Vigorous | 3.83 ± 0.39 | 1.25 ± 0.45** | 2.33 ± 0.49** |
| Uncertain about things | 0.25 ± 0.45 | 2.17 ± 0.39** | 3.25 ± 0.45** |
| Bushed | 0.17 ± 0.39 | 2.33 ± 0.49** | 3.08 ± 0.29** |
| Embarrassed | 0.17 ± 0.39 | 2.42 ± 0.51** | 3.33 ± 0.49** |

**p < 0.001 compared to Composition 2;
^{#}the standard deviations are greater than the means in the negative mood scores is because most of the participants administered with Composition 2 scaled their negative mood states as “0”

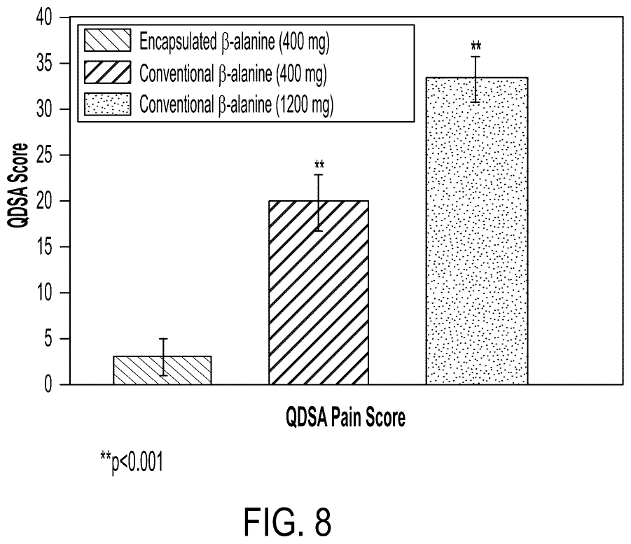

The QDSA pain scores are shown in Table 10 below and FIG. 8. In QDSA, pain scores span from a minimum pain score of 0 to a maximum pain score of 78. The higher the pain score, the greater the pain sensation perceived, which corresponds to the amount of paresthesia experienced by a participant. The data shows that the participants administered with Composition 2 described herein had an average QDSA pain score of 3.33±2.10, indicating no pain after consuming a composition described herein and the same participants administered with reference product I and reference product II had QDSA average pain scores of 19.92±2.94 and 33.33±2.39 respectively, indicating mild and moderate pain respectively related to paresthesia after consuming reference product I and reference product II referenced at the beginning of this Example 4. The results further demonstrate that a composition described herein does not cause paresthesia as compared to free unencapsulated beta-alanine.

TABLE 10

| | 12 subjects | | |
|---|---|---|---|
| | administered with | 12 subjects | 12 subjects |
| | Composition 2 | administered with | administered with |
| | described herein | reference product I | reference product II |
| | containing 400 mg of | containing 400 mg of | containing 1200 mg of |
| | encapsulated beta- | conventional beta- | conventional beta- |
| | alanine | alanine | alanine |
| QDSA pain score | 3.33 ± 2.10 | 19.92 ± 2.94** | 33.33 ± 2.39** |

**p < 0.001

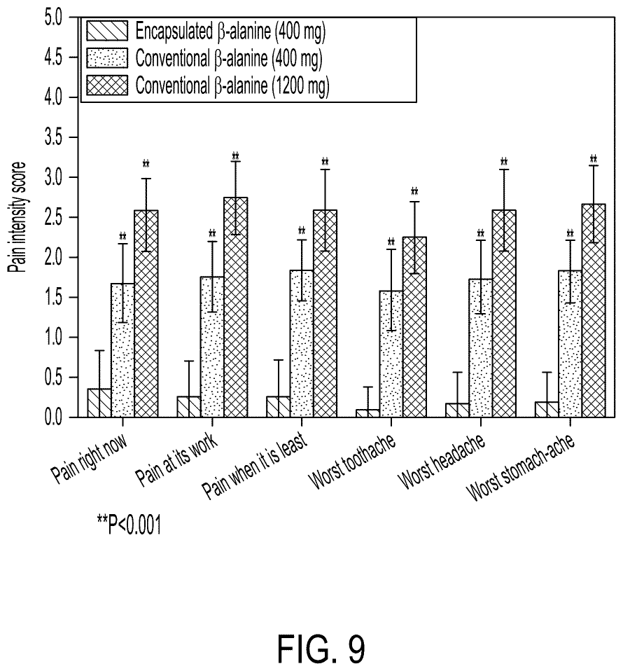

QDSA also included six pain-intensity questions, i.e., pain right now, pain at its worst, pain when it is least, worst toothache, worst headache, and worst stomachache. The pain intensity was scaled from score 0 to score 5, with 0 being no pain and 5 being excruciating pain. The data in Table 11 and FIG. 9 shows that the 12 participants administered with Composition 2 described herein experienced no pain as evidenced by scores ranging from 0.08±0.29 to 0.33±0.49, while the same 12 participants administered with reference product I and reference product II experienced moderate and strong pain respectively as indicated by scores ranging from 1.58±0.51 to 2.75±0.45.

TABLE 11

| | 12 subjects | | |
|---|---|---|---|
| | administered with | 12 subjects | 12 subjects |
| | Composition 2 | administered with | administered with |
| | described herein | reference product I | reference product II |
| | containing 400 mg | containing 400 mg | containing 1200 mg |
| QDSA - Pain | of encapsulated | of conventional | of conventional |
| intensity | beta-alanine^{#} | beta-alanine | beta-alanine |
| Pain right now | 0.33 ± 0.49 | 1.67 ± 0.49** | 2.58 ± 0.51** |
| Pain at its worst | 0.25 ± 0.45 | 1.75 ± 0.45** | 2.75 ± 0.45** |
| Pain when it is least | 0.25 ± 0.45 | 1.83 ± 0.39** | 2.58 ± 0.51** |
| Worst toothache | 0.08 ± 0.29 | 1.58 ± 0.51** | 2.25 ± 0.45** |
| Worst headache | 0.17 ± 0.39 | 1.75 ± 0.45** | 2.58 ± 0.51** |
| Worst stomach-ache | 0.17 ± 0.39 | 1.83 ± 0.39** | 2.67 ± 0.49** |

**p < 0.001 compared to Composition 2;
^{#}the standard deviations are greater than the means is because most of the participants administered with Composition 2 scaled their pain intensity as “0”

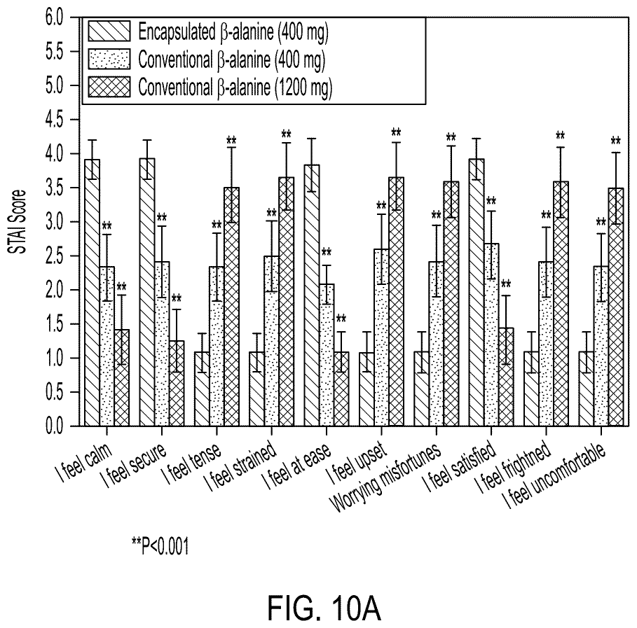

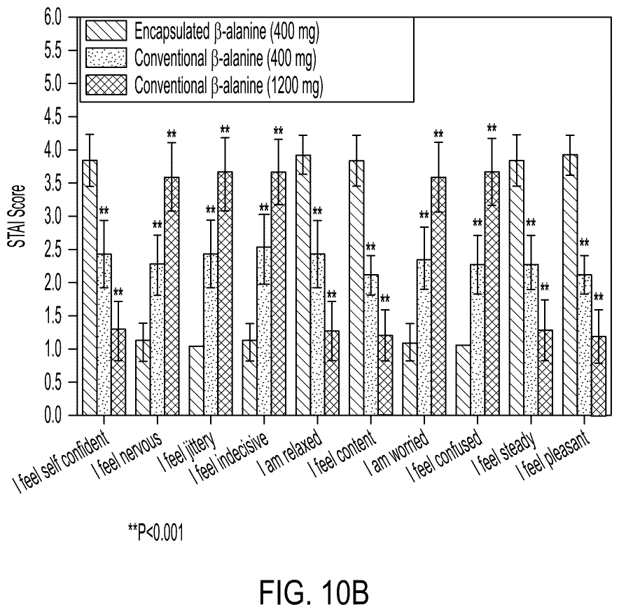

The STAI was used to measure mood and anxiety based on 20 parameters. Mean±SD scores from each group were calculated. Scores 1-4 refer to not at all, a little, somewhat, and very much so, respectively. The results in Table 12 and FIGS. 10A-10B show that the participants administered with Composition 2 described herein scored significantly higher in positive feelings than the same participants administered with reference product I and reference product II, while the participants administered with reference product I and reference product II scored significantly higher in negative feelings than the same participants administered with Composition 2 described herein. The results indicate that a composition described herein can be used to ease anxiety and promote healthy emotions.

TABLE 12

| | 12 subjects | | |
|---|---|---|---|
| | administered with | 12 subjects | 12 subjects |
| | Composition 2 | administered with | administered with |
| | described herein | reference produc tI | reference product II |
| | containing 400 mg | containing 400 mg | containing 1200 mg |
| | of encapsulated | of conventional | of conventional |
| STAI parameters | beta-alanine | beta-alanine | beta-alanine |
| I feel calm | 3.92 ± 0.29 | 2.33 ± 0.49** | 1.42 ± 0.51** |
| I feel secure | 3.92 ± 0.29 | 2.42 ± 0.51** | 1.25 ± 0.45** |
| I feel tense | 1.08 ± 0.29 | 2.33 ± 0.49** | 3.50 ± 0.52** |
| I feel strained | 1.08 ± 0.29 | 2.50 ± 0.52** | 3.67 ± 0.49** |
| I feel at ease | 3.83 ± 0.39 | 2.08 ± 0.29** | 1.08 ± 0.29** |
| I feel upset | 1.08 ± 0.29 | 2.58 ± 0.51** | 3.67 ± 0.49** |
| I am presently worrying over | 1.08 ± 0.29 | 2.42 ± 0.51** | 3.58 ± 0.51** |
| possible misfortunes | | | |
| I feel satisfied | 3.92 ± 0.29 | 2.67 ± 0.49** | 1.42 ± 0.51** |
| I feel frightened | 1.08 ± 0.29 | 2.42 ± 0.51** | 3.58 ± 0.51** |
| I feel uncomfortable | 1.08 ± 0.29 | 2.33 ± 0.49** | 3.50 ± 0.52** |
| I feel self-confident | 3.83 ± 0.39 | 2.42 ± 0.51** | 1.25 ± 0.45** |
| I feel nervous | 1.08 ± 0.29 | 2.25 ± 0.45** | 3.58 ± 0.51** |
| I feel jittery | 1.00 ± 0.00 | 2.42 ± 0.51** | 3.67 ± 0.49** |
| I feel indecisive | 1.08 ± 0.29 | 2.50 ± 0.52** | 3.67 ± 0.49** |
| I am relaxed | 3.92 ± 0.29 | 2.42 ± 0.51** | 1.25 ± 0.45** |
| I feel content | 3.83 ± 0.39 | 2.08 ± 0.29** | 1.17 ± 0.39** |
| I am worried | 1.08 ± 0.29 | 2.33 ± 0.49** | 3.58 ± 0.51** |
| I feel confused | 1.00 ± 0.00 | 2.25 ± 0.45** | 3.67 ± 0.49** |
| I feel steady | 3.83 ± 0.39 | 2.25 ± 0.45** | 1.25 ± 0.45** |
| I feel pleasant | 3.92 ± 0.29 | 2.08 ± 0.29** | 1.17 ± 0.39** |

**p < 0.001 compared to Composition 2

All participants were observed for occurrence of any adverse events throughout the study period. No participant experienced any adverse events, indicating the safety of a composition described herein.

### Example 5: A Double-Blind, Randomized 30-Day Study on the Test Compositions' Effect on Biomarkers and Paresthesia

8 hours after the third treatment from Example 4, the four participants whose third treatment was Composition 2 described herein continued a single dose of Composition 2 each day in the morning with 250 mL of water for 30 consecutive days (“Composition 2 group”) and the four participants whose third treatment was reference product I continued a single dose of reference product I each day in the morning with 250 mL of water for 30 consecutive days (“reference product I group”). Before continuing the dosage, blood samples from each participant (5 mL each) were collected (“Visit 1”). At day 30, blood samples from the participants (5 mL each) were collected again (“Visit 2”). Levels of biomarker lactate, cortisol, total leukocyte count, and C-reactive protein from Visit 1 and Visit 2 were compared. Paresthesia between Visit 1 and Visit 2 and between the two groups was also compared using VAS, QLSI, and Session-dependent questionnaires.

Lactate is an important fuel source for muscles during exercise, including those in the heart. The normal lactate range in human blood is between 4.5 and 19.8 mg/dL. High lactate levels can lead to muscle ache, burning, rapid breathing, nausea, and stomach pain. The results in Table 13 show that Composition 2 did not affect lactate levels and by contrast, reference product I affected lactate levels greatly in some subjects (e.g., in subject 2, lactate increased from 18.0 mg/dL to 26.1 mg/dL, outside of the normal range).

TABLE 13

| Composition 2 Group | | | Reference product I Group | | |
|---|---|---|---|---|---|
| | Visit 1 | Visit 2 | | Visit 1 | Visit 2 |
| Subject | (mg/dL) | (mg/dL) | Subject | (mg/dL) | (mg/dL) |
| 1 | 11.3 | 10.5 | 1 | 18.0 | 26.1 |
| 2 | 12.5 | 12 | 2 | 18.9 | 16.8 |
| 3 | 17.1 | 16.3 | 3 | 18.1 | 15.1 |
| 4 | 29.9 | 29.5 | 4 | 12.4 | 9.6 |
| Average | 17.70 | 17.08 | Average | 16.85 | 16.90 |
| SD | 8.51 | 8.64 | SD | 2.99 | 6.86 |

The results in Table 13 show that the encapsulated beta-alanine exhibited consistent reductions in cortisol, albeit less significant in some instances, than conventional unencapsulated beta-alanine. Notably, the results were not obtained during periods of, or after, physical exertion that would increase lactate concentration. Here, unlike conventional unencapsulated beta-alanine, the encapsulated beta-alanine exhibited no instances of increased lactate content. This result, coupled with the other results contained herein (e.g., improved bioavailability) indicate that encapsulated beta-alanine would provide improved lactate reduction during physical exertion because encapsulated beta-alanine showed no lactate increases from baseline, unlike conventional unencapsulated beta-alanine. Accordingly, experimentation may be performed to further elucidate these benefits.

Cortisol is an essential hormone that affects nearly every organ system in a human body. It regulates the body's stress response, suppresses inflammation, and regulates blood pressure and blood sugar. Normal cortisol levels in humans range from 5 to 25 mcg/dL. High cortisol levels can lead to rapid weight gain, high blood pressure, osteoporosis, muscle weakness, mood changes, and high blood sugar. Low cortisol levels can lead to fatigue, unintentional weight loss, poor appetite, and low blood pressure, so it is desired to maintain baseline levels of cortisol when a subject is not being subjected to stimuli that may raise cortisol levels (e.g., exercise, stress, etc.). The results in Table 14 show that cortisol levels in Composition 2 group changed only slightly after administration of encapsulated beta-alanine, whereas the cortisol levels in reference product I group decreased significantly (e.g., the cortisol level in subject 2 decreased from 11.56 mcg/dL to a low cortisol level of 4.78 mcg/dL) after administration of conventional unencapsulated beta-alanine, further indicating that a composition described herein retains cortisol at baseline levels more than a conventional beta-alanine during the absence of any external cortisol-raising stimulus. This is also beneficial because, for example, maintaining baseline amounts of cortisol can be beneficial when consuming the compositions described herein in the morning, for example, when cortisol levels are increased in the body or before physical or mental exertion when cortisol contributes to mental acuity, alertness, and focus.

TABLE 14

| Composition 2 group | | | Reference product I group | | |
|---|---|---|---|---|---|
| | Visit 1 | Visit 2 | | Visit 1 | Visit 2 |
| Subject | (mcg/dL) | (mcg/dL) | Subject | (mcg/dL) | (mcg/dL) |
| 1 | 9.45 | 7.11 | 1 | 11.9 | 6.46 |
| 2 | 7.45 | 7.23 | 2 | 10.01 | 6.11 |
| 3 | 8.16 | 7.25 | 3 | 11.56 | 4.78 |
| 4 | 5.74 | 7.14 | 4 | 6.01 | 6.07 |
| Average | 7.70 | 7.18 | Average | 9.87 | 5.86 |
| SD | 1.55 | 0.07 | SD | 2.70 | 0.74 |

Leukocytes, commonly known as white blood cells, play a crucial role in the body's immune system. The normal white blood cell count range is between 4,000 and 11,000/μl. High white blood cell count can lead to fatigue, pain, fever, and difficulty breathing. The data in Table 15 shows that leukocytes in Composition 2 group changed slightly, while leukocytes in reference product I group changed significantly (e.g., leukocytes in subjects 1 and 3 increased from 7400 to 13200 and from 5900 to 12900 respectively, much higher than the upper limit of the normal range). These results indicate that the compositions described herein maintain a beneficial baseline amount of leukocytes after ingestion of encapsulated beta-alanine compared to conventional unencapsulated beta-alanine.

TABLE 15

| Composition 2 group | | | Reference product I group | | |
|---|---|---|---|---|---|
| Subject | Visit 1 | Visit 2 | Subject | Visit 1 | Visit 2 |
| 1 | 6100 | 5000 | 1 | 7400 | 13200 |
| 2 | 4400 | 4800 | 2 | 5700 | 4600 |
| 3 | 4800 | 6400 | 3 | 5900 | 12900 |
| 4 | 6900 | 4800 | 4 | 7400 | 6600 |
| Average | 5550.00 | 5250.00 | Average | 6600.00 | 9325.00 |
| SD | 1156.14 | 772.44 | SD | 927.36 | 4379.78 |

C-reactive protein (CPR) is a pentameric protein that increases in response to inflammation. The normal CPR level is less than 3 mg/L. A CPR level of 3 mg/L to 10 mg/L can indicate minor inflammation and a CPR level above 10 mg/L can indicate significant inflammation. The results in Table 16 show that Composition 2 described herein can reduce CPR levels, while a conventional unencapsulated beta-alanine can elevate CPR from normal levels to abnormal levels, which may indicate inflammation. The results demonstrate an unexpected advantage of a composition described herein, wherein the encapsulated beta-alanine can reduce inflammation compared to conventional unencapsulated beta-alanine.

TABLE 16

| Composition 2 group | | | Reference product I group | | |
|---|---|---|---|---|---|
| Subject | Visit 1 | Visit 2 | Subject | Visit 1 | Visit 2 |
| 1 | 1.36 | 1.48 | 1 | 2.55 | 3.62 |
| 2 | 1.21 | 1.19 | 2 | 2.24 | 19.2 |
| 3 | 5.37 | 1.59 | 3 | 4.04 | 0.51 |
| 4 | 3.3 | 1.55 | 4 | 2.07 | 16.23 |
| Average | 2.81 | 1.45 | Average | 2.73 | 9.89 |
| SD | 1.95 | 0.18 | SD | 0.90 | 9.20 |

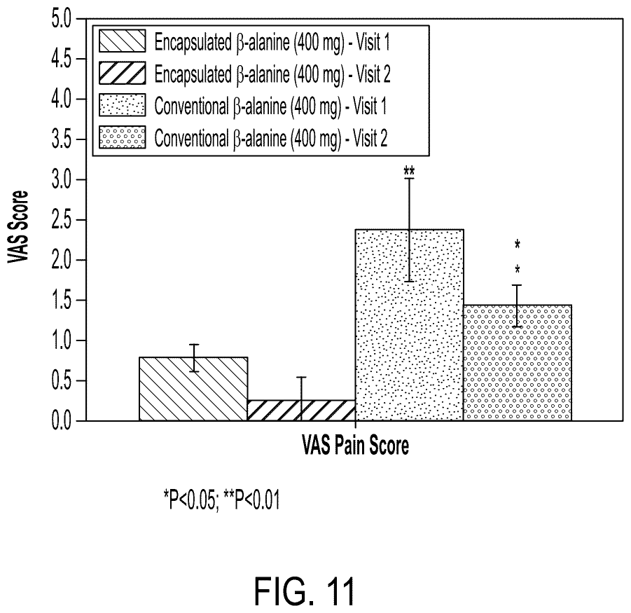

VAS results in Table 17 and FIG. 11 show that, at both Visit 1 and Visit 2, Composition 2 group experienced no sensations associated with paresthesia, and therefore no paresthesia, and reference product I group experienced mild sensations associated with paresthesia. The results again demonstrate that a composition described herein does not cause paresthesia as compared to compositions containing the same amount of beta-alanine. The results also demonstrate that a composition described herein can be used for a long period without causing paraesthesia sensation.

TABLE 17

| Composition 2 group | | Reference product I group | |
|---|---|---|---|
| Visit 1 | Visit 2 | Visit 1 | Visit 2 |
| 0.78 ± 0.17 | 0.25 ± 0.29 | 2.38 ± 0.65** | 1.43 ± 0.26**,* |

*P < 0.05 between the two visits and
**P < 0.01 between the two groups

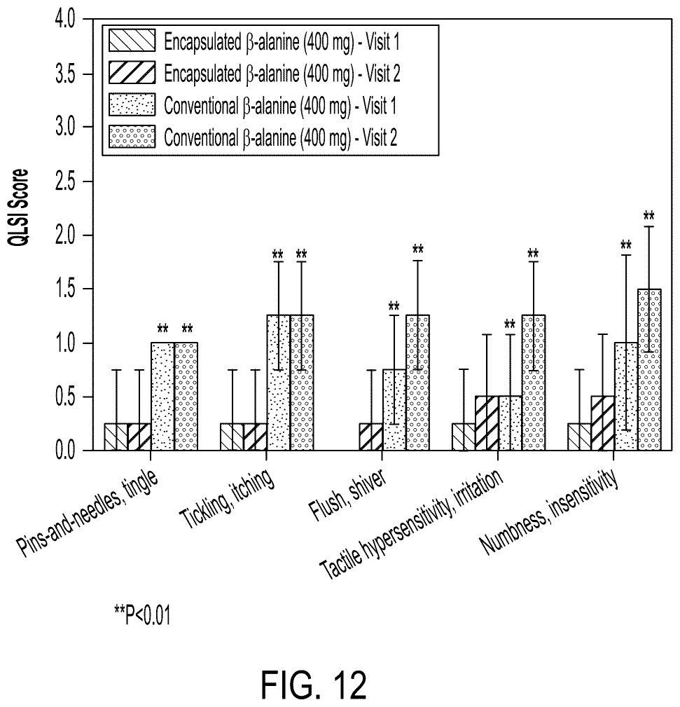

QLSI in Table 18 and FIG. 12 show that (i) the scores in all the sensations recorded by Composition 2 group from Visit 1 and Visit 2 are significantly lower than those in reference product I group and (ii) after 30-day dosing, Composition 2 group experienced nearly no paresthesia sensation changes and by contrast, reference product I group experienced significantly increased paresthesia sensation such as flush/shiver, tactile hypersensitivity/irritation, and numbness/insensitivity. The results further demonstrate that a composition described herein does not cause paresthesia after 30-day dosing as compared to the same amount of free beta-alanine and the compositions described herein are suitable for long-term use.

TABLE 18

| | Composition 2 group^{#} | | Reference product I group | |
|---|---|---|---|---|
| QLSI parameters | Visit 1 | Visit 2 | Visit 1 | Visit 2 |
| Pins-and-needles, tingle | 0.25 ± 0.50 | 0.25 ± 0.50 | 1.00 ± 0.00** | 1.00 ± 0.00** |
| Tickling, itching | 0.25 ± 0.50 | 0.25 ± 0.50 | 1.25 ± 0.50** | 1.25 ± 0.50** |
| Flush, shiver | 0.00 ± 0.00 | 0.25 ± 0.50 | 0.75 ± 0.50** | 1.25 ± 0.50** |
| Tactile hypersensitivity, irritation | 0.25 ± 0.50 | 0.50 ± 0.58 | 0.50 ± 0.58** | 1.25 ± 0.50** |
| Numbness, insensitivity | 0.25 ± 0.50 | 0.50 ± 0.58 | 1.00 ± 0.82** | 1.50 ± 0.58** |
| Pain, soreness | 0.25 ± 0.50 | 0.50 ± 0.58 | 1.25 ± 0.50** | 1.25 ± 0.50** |

**P < 0.01 between the two groups;
^{#}because most of the participants administered with Composition 2 scaled their sensation as “0”, the standard deviations are greater than the means

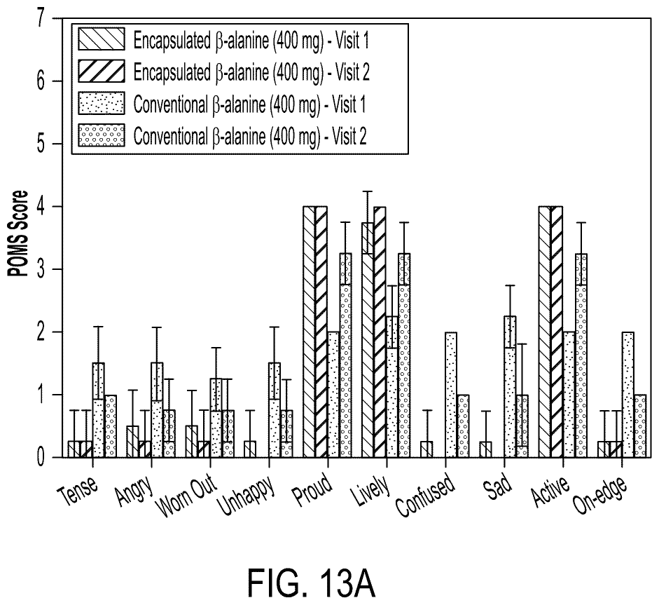

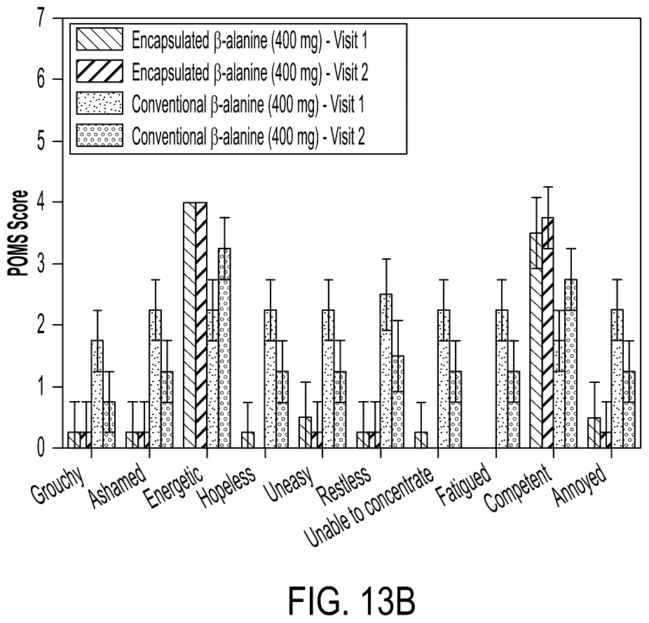

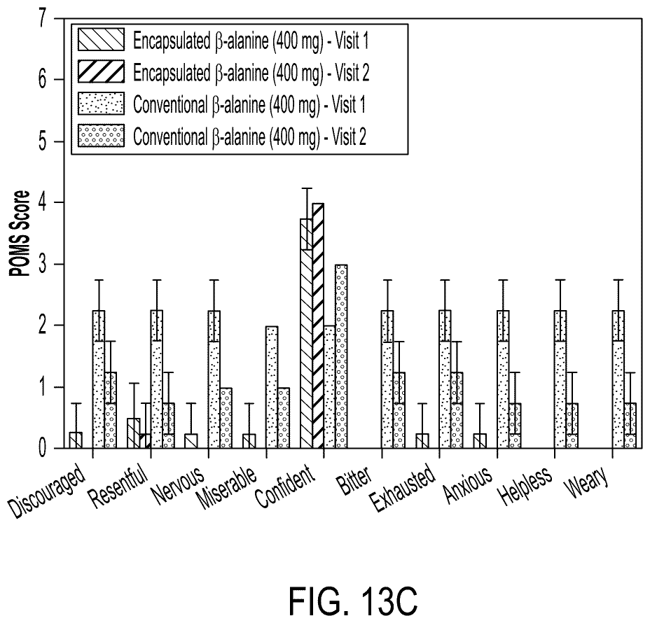

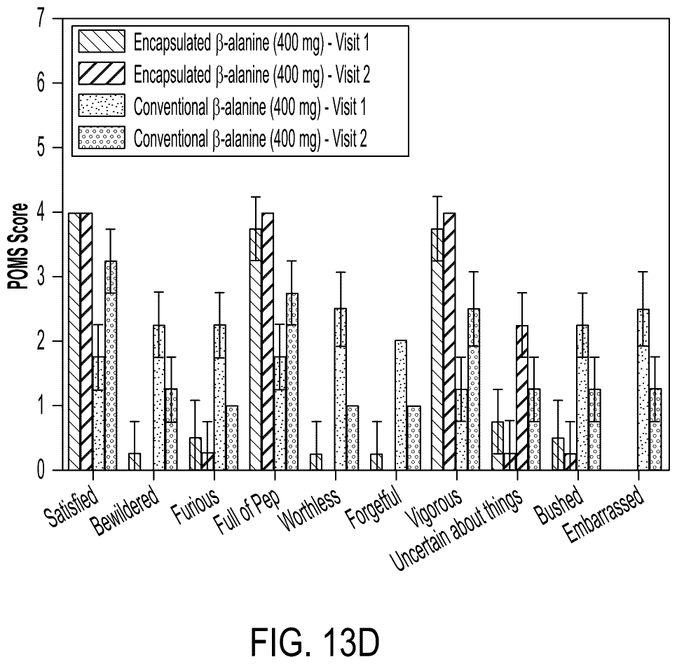

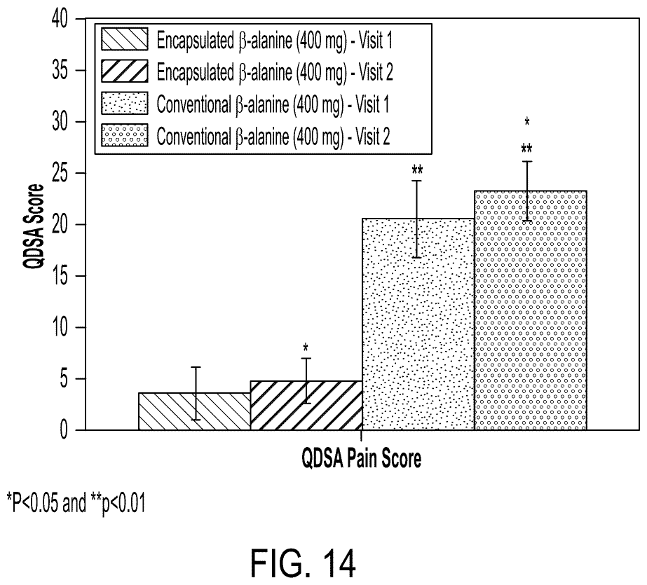

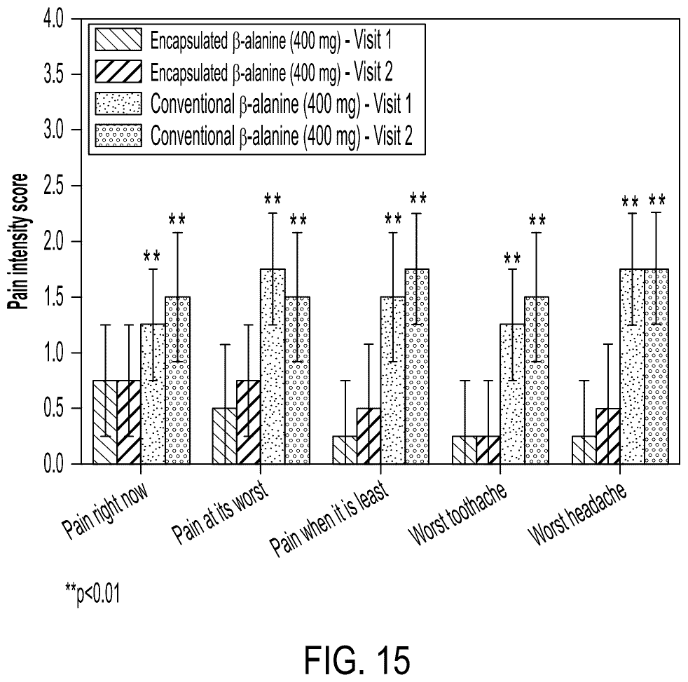

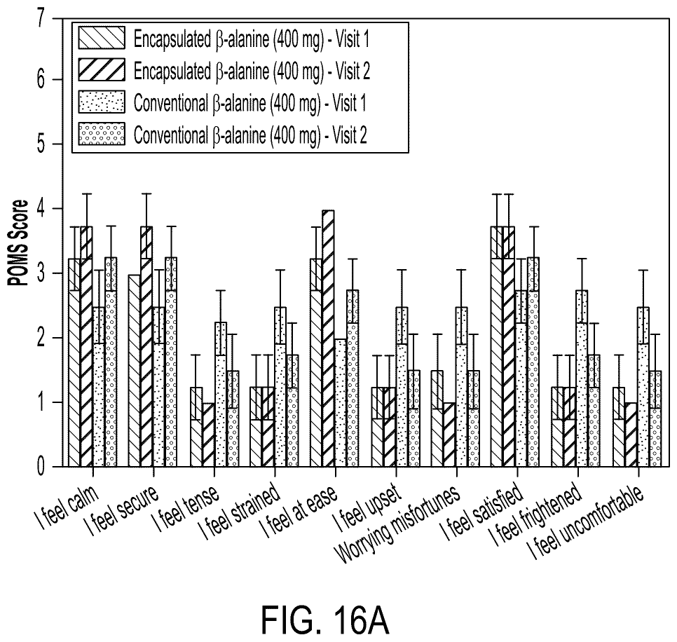

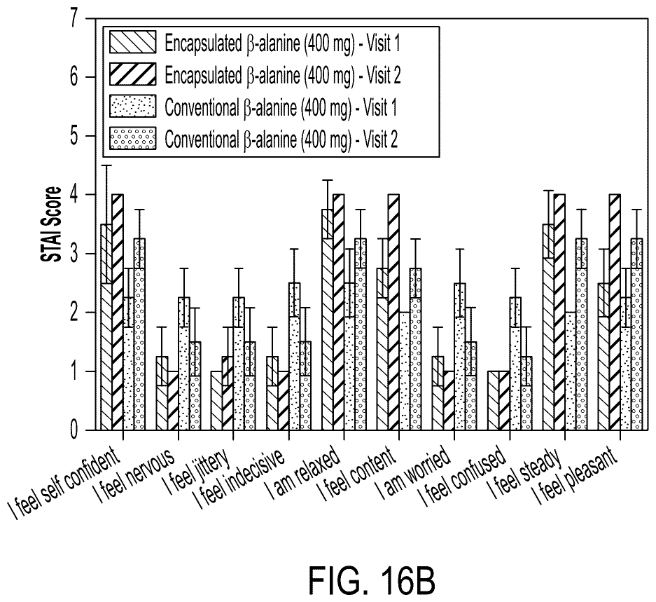

Session-dependent questionnaires results, including POMS, QDSA, QDSA pain intensity, and STAI, are shown in, respectively, Table 19 and FIGS. 13A-13D, Table 20 and FIG. 14, Table 21 and FIG. 15, and Table 22 and FIGS. 16A-16B.

Table 19 and FIGS. 13A-13D show that (i) Composition 2 group scored significantly higher in positive moods (such as proud, active, energetic, competent, confident, satisfied, full of pep, and vigorous) than reference product I group, while reference product I group scored significantly higher in negative moods (such as tense, angry, worn out, unhappy, confused, sad, no-edge, grouchy ashamed, hopeless, uneasy, restless, unable to concentrate, fatigued, annoyed, discouraged, resentful, nervous, miserable, bitter, exhausted, anxious, helpless, weary, bewildered, furious, worthless, forgetful, uncertain about things, bushed, and embarrassed) than Composition 2 group in both Visit 1 and Visit 2; and (ii) in Composition 2 group, there was no significant change between Visit 1 and Visit 2. The results indicate that a composition as described herein is advantageous in promoting a healthy mood over free beta-alanine and can be used to maintain a healthy mood.

TABLE 19

| POMS | Composition 2 group^{#} | | Reference product I group | |
|---|---|---|---|---|
| Parameters | Visit 1 | Visit 2 | Visit 1** | Visit 2** |
| Tense | 0.25 ± 0.50 | 0.25 ± 0.50 | 1.50 ± 0.58 | 1.00 ± 0.00 |
| Angry | 0.50 ± 0.58 | 0.25 ± 0.50 | 1.50 ± 0.58 | 0.75 ± 0.50 |
| Worn Out | 0.50 ± 0.58 | 0.25 ± 0.50 | 1.25 ± 0.50 | 0.75 ± 0.50 |
| Unhappy | 0.25 ± 0.50 | 0.00 ± 0.00 | 1.50 ± 0.58 | 0.75 ± 0.50 |
| Proud | 4.00 ± 0.00 | 4.00 ± 0.00 | 2.00 ± 0.00 | 3.25 ± 0.50 |
| Lively | 3.75 ± 0.50 | 4.00 ± 0.00 | 2.25 ± 0.50 | 3.25 ± 0.50 |
| Confused | 0.25 ± 0.50 | 0.00 ± 0.00 | 2.00 ± 0.00 | 1.00 ± 0.00 |
| Sad | 0.25 ± 0.50 | 0.00 ± 0.00 | 2.25 ± 0.50 | 1.00 ± 0.82 |
| Active | 4.00 ± 0.00 | 4.00 ± 0.00 | 2.00 ± 0.00 | 3.25 ± 0.50 |
| On-edge | 0.25 ± 0.50 | 0.25 ± 0.50 | 2.00 ± 0.00 | 1.00 ± 0.00 |
| Grouchy | 0.25 ± 0.50 | 0.25 ± 0.50 | 1.75 ± 0.50 | 0.75 ± 0.50 |
| Ashamed | 0.25 ± 0.50 | 0.25 ± 0.50 | 2.25 ± 0.50 | 1.25 ± 0.50 |
| Energetic | 4.00 ± 0.00 | 4.00 ± 0.00 | 2.25 ± 0.50 | 3.25 ± 0.50 |
| Hopeless | 0.25 ± 0.50 | 0.00 ± 0.00 | 2.25 ± 0.50 | 1.25 ± 0.50 |
| Uneasy | 0.50 ± 0.58 | 0.25 ± 0.50 | 2.25 ± 0.50 | 1.25 ± 0.50 |
| Restless | 0.25 ± 0.50 | 0.25 ± 0.50 | 2.50 ± 0.58 | 1.50 ± 0.58 |
| Unable to | 0.25 ± 0.50 | 0.00 ± 0.00 | 2.25 ± 0.50 | 1.25 ± 0.50 |
| concentrate | | | | |
| Fatigued | 0.00 ± 0.00 | 0.00 ± 0.00 | 2.25 ± 0.50 | 1.25 ± 0.50 |
| Competent | 3.50 ± 0.58 | 3.75 ± 0.50 | 1.75 ± 0.50 | 2.75 ± 0.50 |
| Annoyed | 0.50 ± 0.58 | 0.25 ± 0.50 | 2.25 ± 0.50 | 1.25 ± 0.50 |
| Discouraged | 0.25 ± 0.50 | 0.00 ± 0.00 | 2.25 ± 0.50 | 1.25 ± 0.50 |
| Resentful | 0.50 ± 0.58 | 0.25 ± 0.50 | 2.25 ± 0.50 | 0.75 ± 0.50 |
| Nervous | 0.25 ± 0.50 | 0.00 ± 0.00 | 2.25 ± 0.50 | 1.00 ± 0.00 |
| Miserable | 0.25 ± 0.50 | 0.00 ± 0.00 | 2.00 ± 0.00 | 1.00 ± 0.00 |
| Confident | 3.75 ± 0.50 | 4.00 ± 0.00 | 2.00 ± 0.00 | 3.00 ± 0.00 |
| Bitter | 0.00 ± 0.00 | 0.00 ± 0.00 | 2.25 ± 0.50 | 1.25 ± 0.50 |
| Exhausted | 0.25 ± 0.50 | 0.00 ± 0.00 | 2.25 ± 0.50 | 1.25 ± 0.50 |
| Anxious | 0.25 ± 0.50 | 0.00 ± 0.00 | 2.25 ± 0.50 | 0.75 ± 0.50 |
| Helpless | 0.00 ± 0.00 | 0.00 ± 0.00 | 2.25 ± 0.50 | 0.75 ± 0.50 |
| Weary | 0.00 ± 0.00 | 0.00 ± 0.00 | 2.25 ± 0.50 | 0.75 ± 0.50 |
| Satisfied | 4.00 ± 0.00 | 4.00 ± 0.00 | 1.75 ± 0.50 | 3.25 ± 0.50 |
| Bewildered | 0.25 ± 0.50 | 0.00 ± 0.00 | 2.25 ± 0.50 | 1.25 ± 0.50 |
| Furious | 0.50 ± 0.58 | 0.25 ± 0.50 | 2.25 ± 0.50 | 1.00 ± 0.00 |
| Full of Pep | 3.75 ± 0.50 | 4.00 ± 0.00 | 1.75 ± 0.50 | 2.75 ± 0.50 |
| Worthless | 0.25 ± 0.50 | 0.00 ± 0.00 | 2.50 ± 0.58 | 1.00 ± 0.00 |
| Forgetful | 0.25 ± 0.50 | 0.00 ± 0.00 | 2.00 ± 0.00 | 1.00 ± 0.00 |
| Vigorous | 3.75 ± 0.50 | 4.00 ± 0.00 | 1.25 ± 0.50 | 2.50 ± 0.58 |
| Uncertain | 0.75 ± 0.50 | 0.25 ± 0.50 | 2.25 ± 0.50 | 1.25 ± 0.50 |
| about things | | | | |
| Bushed | 0.50 ± 0.58 | 0.25 ± 0.50 | 2.25 ± 0.50 | 1.25 ± 0.50 |
| Embarrassed | 0.00 ± 0.00 | 0.00 ± 0.00 | 2.50 ± 0.58 | 1.25 ± 0.50 |

**P < 0.01 between the two groups;
^{#}because most of the participants administered with Composition 2 scaled their negative mood states as “0”, the standard deviations in such negative mood states are greater than the means

QDSA results in Table 20 and FIG. 14 show that Composition 2 group had an average QDSA pain score of 3.50±2.52 at Visit 1 and a slightly increased average QDSA pain score of 4.75±2.22 at Visit 2, indicating no pain after 8 hours of consuming a composition described herein and after 30 days of continuing consumption of the composition and reference product I group had an average QDSA pain score of 20.50±3.79 at Visit 1 and a significant increased average QDSA pain score of 23.25±2.87, indicating mild pain to moderate pain related to paresthesia after 8 hours of consuming reference product I and after 30 days of continuing consuming reference product 1. The results further demonstrate that a composition described herein does not cause paresthesia as compared to free beta-alanine after 30-day consummation and is suitable for long-term use.

TABLE 20

| | |
|---|---|
| Composition 2 group | Reference product I group |

| Visit 1 | Visit 2 | Visit 1 | Visit 2 |
|---|---|---|---|
| 3.50 ± 2.52 | 4.75 ± 2.22* | 20.50 ± 3.79** | 23.25 ± 2.87** |

*P < 0.05 between the two visits and
**P < 0.01 between the two groups

QDSA pain intensity results in Table 21 and FIG. 15 show that in both Composition 2 group and reference product I group, there were no significant changes between Visit 1 and Visit 2 and that Composition 2 group experienced no pain while reference product I group experienced moderate pain. The results again demonstrate that a composition described herein does not cause paresthesia after 30-day dosing and is suitable for long-term use.

TABLE 21

| | Composition 2 group^{#} | | Reference product I group | |
|---|---|---|---|---|
| QDSA - Pain intensity | Visit 1 | Visit 2 | Visit 1 | Visit 2 |
| Pain right now | 0.75 ± 0.50 | 0.75 ± 0.50 | 1.25 ± 0.50** | 1.50 ± 0.58** |
| Pain at its worst | 0.50 ± 0.58 | 0.75 ± 0.50 | 1.75 ± 0.50** | 1.50 ± 0.58** |
| Pain when it is least | 0.25 ± 0.50 | 0.50 ± 0.58 | 1.50 ± 0.58** | 1.75 ± 0.50** |
| Worst toothache | 0.25 ± 0.50 | 0.25 ± 0.50 | 1.25 ± 0.50** | 1.50 ± 0.58** |
| Worst headache | 0.25 ± 0.50 | 0.50 ± 0.58 | 1.75 ± 0.50** | 1.75 ± 0.50** |
| Worst stomach-ache | 0.00 ± 0.00 | 0.25 ± 0.50 | 1.75 ± 0.50** | 1.75 ± 0.50** |

**P < 0.01 between the two groups;
^{#}because most of the participants administered with Composition 2 scaled their pain intensity as “0”, the standard deviations are greater than the means

STAI results in Table 22 and FIGS. 16A-16D show that there were no significant changes between Visit 1 and Visit 2 in both groups and that Composition 2 group scored significantly higher in positive feelings than reference product I group, while reference product I group scored significantly higher in negative feelings than Composition 2 group. The results indicate that a composition described herein is advantageous over free beta-alanine in promoting healthy emotion and can be used to maintain healthy emotions.

TABLE 22

| STAI | Composition 2 group | | Reference product I group | |
|---|---|---|---|---|
| Parameters | Visit 1 | Visit 2 | Visit 1 | Visit 2 |
| I feel calm | 3.25 ± 0.50 | 3.75 ± 0.50 | 2.50 ± 0.58 | 3.25 ± 0.50 |
| I feel secure | 3.00 ± 0.00 | 3.75 ± 0.50 | 2.50 ± 0.58 | 3.25 ± 0.50 |
| I feel tense | 1.25 ± 0.50 | 1.00 ± 0.00 | 2.25 ± 0.50 | 1.50 ± 0.58 |
| I feel strained | 1.25 ± 0.50 | 1.25 ± 0.50 | 2.50 ± 0.58 | 1.75 ± 0.50 |
| I feel at ease | 3.25 ± 0.50 | 4.00 ± 0.00 | 2.00 ± 0.00 | 2.75 ± 0.50 |
| I feel upset | 1.25 ± 0.50 | 1.25 ± 0.50 | 2.50 ± 0.58 | 1.50 ± 0.58 |
| I am presently | 1.50 ± 0.58 | 1.00 ± 0.00 | 2.50 ± 0.58 | 1.50 ± 0.58 |
| worrying over | | | | |
| possible | | | | |
| misfortunes | | | | |
| I feel satisfied | 3.75 ± 0.50 | 3.75 ± 0.50 | 2.75 ± 0.50 | 3.25 ± 0.50 |
| I feel | 1.25 ± 0.50 | 1.25 ± 0.50 | 2.75 ± 0.50 | 1.75 ± 0.50 |
| frightened | | | | |
| I feel | 1.25 ± 0.50 | 1.00 ± 0.00 | 2.50 ± 0.58 | 1.50 ± 0.58 |
| uncomfortable | | | | |
| I feel self- | 3.50 ± 1.00 | 4.00 ± 0.00 | 2.25 ± 0.50 | 3.25 ± 0.50 |
| confident | | | | |
| I feel nervous | 1.25 ± 0.50 | 1.00 ± 0.00 | 2.25 ± 0.50 | 1.50 ± 0.58 |
| I feel jittery | 1.00 ± 0.00 | 1.25 ± 0.50 | 2.25 ± 0.50 | 1.50 ± 0.58 |
| I feel | 1.25 ± 0.50 | 1.00 ± 0.00 | 2.50 ± 0.58 | 1.50 ± 0.58 |
| indecisive | | | | |
| I am relaxed | 3.75 ± 0.50 | 4.00 ± 0.00 | 2.50 ± 0.58 | 3.25 ± 0.50 |
| I feel content | 2.75 ± 0.50 | 4.00 ± 0.00 | 2.00 ± 0.00 | 2.75 ± 0.50 |
| I am worried | 1.25 ± 0.50 | 1.00 ± 0.00 | 2.50 ± 0.58 | 1.50 ± 0.58 |
| I feel confused | 1.00 ± 0.00 | 1.00 ± 0.00 | 2.25 ± 0.50 | 1.25 ± 0.50 |
| I feel steady | 3.50 ± 0.58 | 4.00 ± 0.00 | 2.00 ± 0.00 | 3.25 ± 0.50 |
| I feel pleasant | 2.50 ± 0.58 | 4.00 ± 0.00 | 2.25 ± 0.50 | 3.25 ± 0.50 |

The biomarker results demonstrate that a composition described herein does not cause adverse effects such as oxidative stress and inflammation compared to free beta-alanine. The parenthesis results demonstrate that a composition described herein is suitable for long-term use without causing paresthesia and can be used to maintain healthy mood and emotions.

### Example 6: Effect on Carnosine Concentrations in Skeletal Muscle, Heart, and Brain

A composition described herein will be assessed for its effect on skeletal muscle, heart, and brain carnosine concentrations.

A clinical trial will be performed to (i) determine the efficacy of the administration of encapsulated beta-alanine compositions described herein at raising skeletal muscle, heart, and brain carnosine concentrations. The clinical trial will constitute at least three clinical arms; one placebo comparator arm, at least one comparator arm wherein the participants receive conventional beta-alanine known in the prior art, and at least one experiment arm wherein the participants receive a beta-alanine composition described herein. Regarding efficacy, the clinical data will demonstrate that the treatment(s) evaluated in the experimental arm(s) has/have an equal or better efficacy than the treatment(s) evaluated in the comparator arm. The clinical data will also demonstrate that the treatments evaluated in the experimental arm and comparator arm both have a better efficacy than the placebo.

### Example 7: Effect on Physical Performance and Endurance

A randomized double blind, placebo-controlled study will be performed to evaluate the effect of a composition described herein on physical performance and endurance.

60 healthy volunteers aged 18-40 will be randomly divided into 3 groups with 20 in each group. In the morning, 30 minutes before a workout, once a day for 8 weeks, Group I will orally take a placebo, Group II will orally take a beta-alanine composition described herein, and Group III will orally take a conventional beta-alanine composition known in the prior art. From day 1 to day 28, each day before and after supplementation, each volunteer will perform an exhaustive exercise on treadmill at 70% of VO2 max. Before and after the exercise, a salivary sample will be collected to assess changes in nitric oxide concentration, a venous blood sample will be collected to assess changes in lactate concentration, beta-alanine concentration, and carnosine concentration, and a single muscle biopsy of the vastus lateralis will be taken to assess changes in muscle carnosine content. Rate of perceived exertion, time to exhaustion, hand grip strength, fat mass/total lean soft tissue mass/upper and lower arm lean soft tissue mass, and quadriceps muscle torque will be compared before and after the exercise to assess the effect of a composition described herein on endurance and physical performance and compared to the effect of placebo and the conventional beta-alanine composition.

Safety will be determined based on adverse events, including paresthesia, nausea, vomiting, headache, fever, and body pain, reported by the participants. Hematology and biochemistry parameters, i.e., hemoglobin, platelet count, RBC, ESR, total WBC count, neutrophils, lymphocytes, eosinophils, monocytes, basophils, fasting blood glucose, serum creatinine, blood urea nitrogen, blood uric acid, serum sodium, serum potassium, serum alkaline phosphate, serum glutamic oxaloacetic transaminase, serum glutamic-pyruvic transaminase, bilirubin, and serum albumin, will be measured from day 1 to day 28.

### Example 8: Effect on Cognitive Function, Mood, and Physical Performance in Elderly

A randomized double blind, placebo-controlled study will be conducted to assess efficacy of a composition described herein on cognitive function, mood, and physical performance in elderly.

A clinical trial will be performed to (i) determine the efficacy of the administration of encapsulated beta-alanine compositions described herein at improving cognitive function, mood, and physical performance amongst elderly (e.g., greater than 55 years old). The clinical trial will constitute at least three clinical arms; one placebo comparator arm, at least one comparator arm wherein the participants receive conventional beta-alanine known in the prior art, and at least one experiment arm wherein the participants receive a beta-alanine composition described herein. Regarding efficacy, the clinical data will demonstrate that the treatment(s) evaluated in the experimental arm(s) has/have an equal or better efficacy than the treatment(s) evaluated in the comparator arm. The clinical data will also demonstrate that the treatments evaluated in the experimental arm and comparator arm both have a better efficacy than the placebo.

The present disclosure is not to be limited in terms of the particular embodiments described in this application, which are intended as illustrations of various aspects of the present disclosure. Many modifications and variations can be made without departing from its spirit and scope. Functionally equivalent methods and apparatuses within the scope of the disclosure, in addition to those enumerated herein, are possible from the foregoing descriptions. Such modifications and variations are intended to fall within the scope of the appended claims. The terminology used herein is for the purpose of describing particular embodiments only and is not intended to be limiting unless otherwise set forth herein.

1. A composition comprising:
at least one compound selected from the group consisting of beta-alanine, an ester of beta-alanine, and a salt of beta-alanine;
an anti-inflammatory agent;
an amphiphilic agent; and
a stimulant.

2. The composition of claim 1, wherein the composition comprises:
at least about 1 wt. % of the at least one compound selected from the group consisting of beta-alanine, an ester of beta-alanine, and a salt of beta-alanine;
at most about 99 wt. % of the anti-inflammatory agent,
at most about 99 wt. % of the amphiphilic agent, and
at most about 99 wt. % of the stimulant.

3. The composition of claim 2, wherein the composition comprises:
between about 10 wt. % and about 99 wt. % of the at least one compound selected from the group consisting of beta-alanine, an ester of beta-alanine, and a salt of beta-alanine;
between about 0.01 wt. % and about 50 wt. % of the anti-inflammatory agent;
between about 0.01 wt. % and about 50 wt. % of the amphiphilic agent; and
between about 0.01 wt. % and about 50 wt. % of the stimulant.

4. The composition of claim 3, wherein the composition comprises:
between about 50 wt. % and about 80 wt. % of the at least one compound selected from the group consisting of beta-alanine, an ester of beta-alanine, and a salt of beta-alanine;
between about 0.01 wt. % and about 10 wt % of the anti-inflammatory agent;
between about 0.1 wt. % and about 10 wt. % of the amphiphilic agent; and
between about 0.01 wt. % and about 10 wt. % of the stimulant.

5. The composition of claim 4, wherein the composition comprises:
between about 70 wt. % and about 80 wt. % of the at least one compound selected from the group consisting of beta-alanine, an ester of beta-alanine, and a salt of beta-alanine;
between about 0.1 wt. % and about 4 wt. % of the anti-inflammatory agent;
between about 1 wt. % and about 10 wt. % of the amphiphilic agent; and
between about 0.1 wt. % and about 1 wt. % of the stimulant.

6. The composition of claim 5, wherein the anti-inflammatory agent is beta-caryophyllene.

7. The composition of claim 1, wherein the stimulant is caffeine.

8. The composition of claim 1, wherein the amphiphilic agent is a phospholipid.

9. The composition of claim 8, wherein the phospholipid is lecithin.

10. The composition of claim 1, wherein the composition is coated with a coating agent.

11. The composition of claim 10, wherein the coating agent is a modified food starch.

12. The composition of claim 1, wherein the composition is formulated as a pharmaceutical composition or a dietary supplement.

13. The composition of claim 5, wherein the composition comprises:
between about 70 wt. % and about 73 wt. % of the at least one compound selected from the group consisting of beta-alanine, an ester of beta-alanine, and a salt of beta-alanine;
between about 0.5 wt. % and about 1 wt. % of the anti-inflammatory agent;
between about 1 wt. % and about 5 wt. % of the amphiphilic agent; and
between about 0.1 wt. % and about 0.2 wt. % of the stimulant.

14. The composition of claim 1, wherein the composition is formulated as a human dietary supplement.

15. The composition of claim 14, wherein the at least one compound selected from the group consisting of beta-alanine, an ester of beta-alanine, and a salt of beta-alanine is provided in an amount ranging from about 0.02 g to about 10 g per serving.

16. The composition of claim 14, wherein the at least one compound selected from the group consisting of beta-alanine, an ester of beta-alanine, and a salt of beta-alanine is provided in an amount ranging from about 0.03 g to about 6.4 g per serving.

17. The composition of claim 14, wherein the at least one compound selected from the group consisting of beta-alanine, an ester of beta-alanine, and a salt of beta-alanine is provided in an amount ranging from about 0.04 g to about 3.2 g per serving.

18. The composition of claim 14, wherein the at least one compound selected from the group consisting of beta-alanine, an ester of beta-alanine, and a salt of beta-alanine is provided in an amount ranging from about 0.05 g to about 2.24 g per serving.

19. The composition of claim 14, wherein the at least one compound selected from the group consisting of beta-alanine, an ester of beta-alanine, and a salt of beta-alanine is provided in an amount ranging from about 0.1 g to about 1 g per serving.

20. The composition of claim 14, wherein the at least one compound selected from the group consisting of beta-alanine, an ester of beta-alanine, and a salt of beta-alanine is provided in an amount ranging from about 0.2 g to about 0.8 g per serving.

21. A method for increasing athletic performance and/or improving cognitive function without causing paresthesia in a subject, the method comprising:
administering an effective amount of a composition to the subject, wherein the composition comprises:
at least one compound selected from the group consisting of beta-alanine, an ester of beta-alanine, and a salt of beta-alanine; and
at least one agent selected from the group consisting of an anti-inflammatory agent and an amphiphilic agent.

22. The method of claim 21, wherein the at least one compound selected from the group consisting of beta-alanine, an ester of beta-alanine, and a salt of beta-alanine in the composition is provided in an amount ranging from 0.02 g to about 10 g per day.

23. The method of claim 21, wherein the at least one compound selected from the group consisting of beta-alanine, an ester of beta-alanine, and a salt of beta-alanine in the composition is provided in an amount ranging from about 0.05 g to about 2.24 g per day.

24. The method of claim 21, wherein the at least one compound selected from the group consisting of beta-alanine, an ester of beta-alanine, and a salt of beta-alanine in the composition is provided in an amount ranging from about 0.1 g to about 1 g per day.

25. The method of claim 21, wherein the at least one compound selected from the group consisting of beta-alanine, an ester of beta-alanine, and a salt of beta-alanine in the composition is provided in an amount ranging from about 0.2 g to about 0.8 g per day.

26. The method of claim 21, wherein the composition further comprises a stimulant.

27. The method of claim 26, wherein the composition comprises:
at least about 1 wt. % of the at least one compound selected from the group consisting of beta-alanine, an ester of beta-alanine, and a salt of beta-alanine;
at most about 99 wt. % of the anti-inflammatory agent;
at most about 99 wt. % of the amphiphilic agent; and
at most about 99 wt. % of the stimulant.

28. The method of claim 27, wherein the composition comprises:
between about 10 wt. % and about 99 wt. % of the at least one compound selected from the group consisting of beta-alanine, an ester of beta-alanine, and a salt of beta-alanine;
between about 0.01 wt. % and about 50 wt. % of the anti-inflammatory agent;
between about 0.01 wt. % and about 50 wt. % of the amphiphilic agent; and
between about 0.01 wt. % and about 50 wt. % of the stimulant.

29. The method of claim 28, wherein the composition comprises:
between about 50 wt. % and about 80 wt. % of the at least one compound selected from the group consisting of beta-alanine, an ester of beta-alanine, and a salt of beta-alanine;
between about 0.01 wt. % and about 10 wt. % of the anti-inflammatory agent;
between about 0.1 wt. % and about 10 wt. % of the amphiphilic agent; and
between about 0.01 wt. % and about 10 wt. % of the stimulant.

30. A method of making a beta-alanine composition, the method comprising:
step (a) adding an amphiphilic agent to water to form a first solution;
step (b) homogenizing the first solution to form a first emulsion;
step (c) adding an anti-inflammatory agent to the first emulsion to form a second solution;
step (d) homogenizing the second solution to form a second emulsion;
step (e) adding beta-alanine, a salt of beta-alanine, and/or an ester of beta-alanine into the second emulsion to form a third solution; and
step (f) homogenizing the third solution to form a third emulsion.
